SEQUENCE LISTING (16 sequences)

SEQ ID NO: 1 - DNA, 612 bp, Homo sapiens
ccaagaagatcccacagcagcttccgaaggcctggatgtgatggcatcacagaagagaccctcacagcgacacggatccaagtacttggccacagcaagtaccatggaccatgcccggcatggcttcctcccaaggcacagagacacgggcatccttgactccatcgggcgcttctttagcggtgacaggggtgcgcccaagcggggctctggcaaggactcacacacaagaactacccactacggctccctgccccagaagtcgcagaggacccaagatgaaaacccagtagtccacttcttcaagaacattgtgacacctcgtacaccccctccatcccaaggaaaggggagaggcctgtccctcagcagatttagctggggaggaagagacagccgctctggatctcccatggcaagacgctgagagcctccctgctcagccttcccgaatcctgccctcggcttcttaatataactgccttaaacgtttaattctacttgcaccaaatagctagttagagcagaccctctcttaatcccgtggggctgtgaacgcggcgggccagcccacggcaccctgactggctaaaactgtttgtccctttttat

SEQ ID NO: 2 - DNA, 2139 bp, Homo sapiens
gaaaacagtgcagccacctccgagagcctggatgtgatggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgccaaagcggggctctggcaaggactcacaccacccggcaagaactgctcactatggctccctgccccagaagtcacacggccggacccaagatgaaaaccccgtagtccacttcttcaagaacattgtgacgcctcgcacaccacccccgtcgcagggaaaggggagaggactgtccctgagcagatttagctggggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctgaaaacccacctggttccggaatcctgtcctcagcttcttaatataactgccttaaaactttaatcccacttgcccctgttacctaattagagcagatgacccctcccctaatgcctgcggagttgtgcacgtagtagggtcaggccacggcagcctaccggcaatttccggccaacagttaaatgagaacatgaaaacagaaaacggttaaaactgtccctttctgtgtgaagatcacgttccttcccccgcaatgtgcccccagacgcacgtgggtcttcagggggccaggtgcacagacgtccctccacgttcacccctccacccttggactttcttttcgccgtggctcggcacccttgcgcttttgctggtcactgccatggaggcacacagctgcagagacagagaggacgtgggcggcagagaggactgttgacatccaagcttcctttgtttttttttcctgtccttctctcacctcctaaagtagacttcatttttcctaacaggattagacagtcaaggagtggcttactacatgtgggagctttttggtatgtgacatgcgggctgggcagctgttagagtccaacgtggggcagcacagagagggggccacctccccaggccgtggctgcccacacaccccaattagctgaattcgcgtgtggcagagggaggaaaaggaggcaaacgtgggctgggcaatggcctcacataggaaacagggtcttcctggagatttggtgatggagatgtcaagcaggtggcctctggacgtcaccgttgccctgcatggtggccccagagcagcctctatgaacaacctcgtttccaaaccacagcccacagccggagagtccaggaagacttgcgcactcagagcagaagggtaggagtcctctagacagcctcgcagccgcgccagtcgcccatagacactggctgtgaccgggcgtgctggcagcggcagtgcacagtggccagcactaaccctccctgagaagataaccggctcattcacttcctcccagaagacgcgtggtagcgagtaggcacaggcgtgcacctgctcccgaattactcaccgagacacacgggctgagcagacggcccctgtgatggagacaaagagctcttctgaccatatccttcttaacacccgctggcatctcctttcgcgcctccctccctaacctactgacccaccttttgattttagcgcacctgtgattgataggccttccaaagagtcccacgctggcatcaccctccccgaggacggagatgaggagtagtcagcgtgatgccaaaacgcgtcttcttaatccaattctaattctgaatgtttcgtgtgggcttaataccatgtctattaatatatagcctcgatgatgagagagttacaaagaacaaaactccagacacaaacctccaaatttttcagcagaagcactctgcgtcgctgagctgaggtcggctctgcgatccatacgtggccgcacccacacagcacgtgctgtgacgatggctgaacggaaagtgtacactgttcctgaatattgaaataaaacaataaactttt

SEQ ID NO: 3 - DNA, 581 bp, Homo sapiens
taatatctagggktttgactctgacccgtgttggggctctcacttcatggcttctcacgcttgtgctgcatatcccacaccaattagacccaaggatcagttggaagtttccaggacatcttcattttatttccaccctcaatccacatttccagatgtctctgcagcaaagcgaaattccaggcaagccttagggaaaaaaggaaaaacaaagaaaatgaaacaattggcagtgaaaggcagaaagagaagatggagcccttagagaagggagtatccctgagtaggtggggaaaaggggaggagaaggggaggaggagaggaggaggaaagcaggcctgtccctttaagggggttggctgtcaatcagaaagcccttttcattgcaggagaagaggacaaagatactcagagagaaaaagtaaaagaccgaagaaggaggctggagagaccaggatccttccagctgaacaaagtcagccacaaagcagactagccagccggctacaattggagtcagagtcccaaagacatgggtaagtttcaaaaactttagcattgaagattcaagaggacacagg

SEQ ID NO: 4 - DNA, 1762 bp, Homo sapiens
ctgctttcagagcctgtgacttcttgtgtgcctctcctgtttctcagcaacatggcatagggcctgggataccaggtctggggatctcagggactcttagcactttaagacacatgtgttcccaggccctggtgtgttcctctagtgccagaaagatgtttcatgctttgctgactttgtataaagtctgtttgtagctgttttgacagaatctcagcgtataactgagggtggggacattagccaagctgcattataggaggacaaaactgccatacaaagtgtccaaaatcattaagcctgcatttttattattgggagtaatatcaaacctcctattttccaattttcatttcttgtcctgtgctagctccatcctgtttggactgctcctcccatatgtaaactaagaagaatcaagcattctttgcaacaaatacacacgatgctcaaaaatgtccaggagcatccaatttccaaagtttcctccacctggaatgctcttcatgctaaaatcctgtctgacaataccagcatctctggcctgcactcatcccttcctggaactccaagtgcatttaccctctgttaccacttacttggctgcctgaattgttagttgaaaatattaggtctacttagctaattcttcctcaggaaattaaagactcccatatggcagagtctgtgtcttttctctcttcatatcccgtataacacccagcataatgctgggcatatagtgagtattccataaatagttgatgaatgactaaaataagcaagcaaacaaacagactagaacaataagaaagaagggactggatttcataatctctctggcttgctatttgaattgctgaattattattatttattaaatattttttaaattctggcaataaaaggtaaggatttattttctttctttctttttttttttcttgagacagagtctcgctcttactgcccaggctggagtacaatggcgcaatcttggctcacggcaacctccgcctcctcctgggtttaacagattctcctgtctcagcctcctgagtagctgggattacaggcatacgcccatgcccggctaatttttgtatttttagtagagacggggttttgccatgttggccaggctggtcttgaactcctgacctcatgtgatccacctgcctcagcctcccaaagtgctgggattacaggcatgcgccaccgtgcccggccaaagatttattttcaagaatgaaacaaagtaaggattctgggtcaatctcacatgctgaaagccaaaacctctagccgctcctgctttttgacttcggagtgcccactatctccgagcctgtgagcacagggcctggcagaggggtttgagtggcatgagctacctactggatgtgcctgactgtttccccttcttcttccccaggcttgttagagtgctgtgcaagatgtctggtaggggccccctttgcttccctggtggccactggattgtgtttctttggggtggcactgttctgtggctgtggacatgaagccctcactggcacagaaaagctaattgagacctatttctccaaaaactaccaagactatgagtatctcatcaatgtgtaagtacctgccctcccacacagacccatcttttttttccctctctccatcctggagatagagaactcttcagtaccttagtaactagcaggggactggggtggagccagaccggattcccgagtcttccctctgtgca

SEQ ID NO: 5 - DNA, 828 bp, Homo sapiens
ctagaaaatccctagccttgttaaggtgctcgctctggtgtatacctcacttatgtcgggaaagaagccaggtcttcaattaataagattccctggtctcgtttgtctacctgttaatgcaggatccatgccttccagtatgtcatctatggaactgcctctttcttcttcctttatggggccctcctgctggctgagggcttctacaccaccggcgcagtcaggcagatctttggcgactacaagaccaccatctgcggcaagggcctgagcgcaacggtaacagggggccagaaggggaggggttccagaggccaacatcaagctcattctttggagcgggtgtgtcattgtttgggaaaatggctaggacatcccgacaaggtgatcatcctcaggattttgtggcaataacaaggggtgggggaaaattgggcgcgagtctgtggcctcgtccccacccaaggctgggtcctctctaggggcctggcatttgagtgaggaagcgatggctgcagccgaacgagaaggtcaggaagaacgtggtgcccagctggcttagcctcacctttcaaaggttccctaagcaaatttcttctcaaaacagaaagcatgagttttgtgggatgctttgtacaatcagaccatttctaagccatctgttggtatccctttgttcccttcctagtaggtaccacaagagtggatctaactggacaagagtctaaaatgctgctcatgtgattgagacttgggcacctgagctragagggaggatggataataaaaattaaataataactccaaggtaaatttacaatgttctgg

SEQ ID NO: 6 - DNA, 1140 bp, Homo sapiens
gatcctcctcattcttcccctacccattccccccaccctccgttatactggggccagttatctagtagatactgccaattacccttggcagaggtgccctgctcactaattttatttgggggagmgccctggaacctggttttaatgtctggcacacgccacttccaggatctcccagtttgtgtttctacatctgcaggctgatgctgatttctaaccaacccatgtcaatcattttagtttgtgggcatcacctatgccctgaccgttgtgtggctcctggtgtttgcctgctctgctgtgcctgtgtacatttacttcaacacctggaccacctgccagtctattgccttccccagcaagacctctgccagtataggcagtctctgtgctgatgccagaatgtatggtgagttagggtacgggtgctttggctctcctacccactatggaagcactatatatttggttattttcttagtgtaaggagggtggtgattatgagaaaaatataagatgatgaatgattgggtcttagtttattaatccttccctactgaaaccagagaggtttcttcccccggaagggaacttggaagtggtgggagttttcttggccattcacattggcctactctagttgactgctgttcacaaccccaaagcagcacatttcaataacaaacacaaggttdsaccactgttcaataccaccttctcttttttgtaaacctgtagaaaagaggatcctaattgttggtagmatccaamtttacagccaggataattagagatggaagaagggctctgggggaaagtctccatgtggccccgtaactccataaagcttaccctgcttgctttttgtgtcttacttaggtgttctcccatggaatgctttccctggcaaggtttgtggctccaaccttctgtccatctgcaaaacagctgaggtgagtgggttatttgggttattttacaagggagtagctaataccatacaaattacacccatggccttcaattttaaggactgaaagtttccctttgctggattttgaattagccgattgccttctacaacatgttggctaagtgtgcctgagccaatgagcatagaaggtaaaacacctcttttct

SEQ ID NO: 7 - DNA, 295 bp, Homo sapiens
  misc_feature (42)..(43): N at positions 42 and 43 is unknown
aattagcacacagaaaggatatccaacacatacaaagctgtnntcatggactacactggagcatattactgctgttgcaagaaacatttcttcttcctcttttcattttcctgcagttccaaatgaccttccacctgtttattgctgcatttgtgggggctgcagctacactggtttccctggtgagttgactttgaatgatcttggcaagtaaataggcctgagatagttgtgggtacagctattctgaaaggcaagaaggtagactgcttccatccttgaaatgctggaggga

SEQ ID NO: 8 - DNA, 2940 bp, Homo sapiens
aattctatatactatcactatggctccactttggatactctccagtggatttagttactcatatggaaatacctgggaggacctcctaacattattagaattgttatgattataatacaaygctatgtcccaggtcttgctgatagtgctacagtgccctgtgaatgtagtgtgctcattgtgcagattaaaaacctaaggcactgaagggtgaagtgatttatctgaagttattttataaagcagtgatcagacaasctgagctcacagaactccctggcccctactgctgaggtttccatacagagtcaagtaatttctcaccttgtaaaacgaattgattcattaaccaggggagagctctactgcatgatgtggctgtgtgtctacagcaagcaccctatgactctaagtcactcggacatattgatgtggcaaagcccaaatattgttcacttccctgaggaaaactcagtgctagatcaaacagaggtgtggaataaatctttatgatttgattctctgggcctgggccatgagacccatgatgcctcagagacatcggacttccagtcaagtgtatatggagaaagccaagcctgggatgtactgctttttgcagagcatgggtttttcccttatttagttatgattttatttctacccttcctcattcccaaagggatttgaggagggagtgctttcttttctactctcattcacattctctcttctgttccctacagctcaccttcatgattgctgccacttacaactttgccgtccttaaactcatgggccgaggcaccaagttctgatcccccgtagaaatccccctttctctaatagcgaggctctaaccacacagcctacaatgctgcgtctcccatcttaactctttgcctttgccaccaactggccctcttcttacttgatgagtgtaacaagaaaggagagtcttgcagtgattaaggtctctctttggactctcccctcttatgtacctcttttagtcattttgcttcatagctggttcctgctagaaatgggaaatgcctaataatatgacttcccaactgcaagtcacaaaggaatggaggctctaattgaattttcaagcatctcctgaggatcagaaagtaatttcttctcaaagggtacttccactgatggaaacaaagtggaaggaaagatgctcaggtacagagaaggaatgtctttggtcctcttgccatctataggggccaaatatattctctttggtgtacaaaatggaattcattctgcgtctctctattacactgaagatagaagaaaaaagaatgtcagaaaaacaataagagcgtttgcccaaatctgcctattgcagctgggagaagggggtcaaagcaaggatctttcacccacagaaagagagcactgaccccgatggcgatggactactgaagccctaactcagccaaccttacttacagcataagggagcgtagaatctgtgtagacgaagggggcatctggccttacacctcgttagggaagagaaacagggtcttgtcagcatcttctcactcccttctccttgataacagctaccatgacaaccctgtggtttccaaggagctgagaatagaaggaaactagcttacatgagaacagactggcctgaggagcagcagttgctggtggctaatggtgtaacctgagatggccctctggtagacacaggatagataactctttggatagcatgtctttttttctgttaattagttgtgtactctggcctctgtcatatcttcacaatggtgctcatttcatggggtattatccattcagtcatcgtaggtgatttgaaggtcttgatttgttttagaatgatgcacatttcatgtattccagtttgtttattacttatttggggttgcatcagaaatgtctggagaataattctttgattatgactgttttttaaactaggaaaattggacattaagcatcacaaatgatattaaaaattggctagttgaatctattgggattttctacaagtattctgcctttgcagaaacagatttggtgaatttgaatctcaatttgagtaatctgatcgttctttctagctaatggaaaatgattttacttagcaatgttatcttggtgtgttaagagttaggtttaacataaaggttattttctcctgatatagatcacataacagaatgcaccagtcatcagctattcagttggtaagcttccagtcatcagctattcagttggtaagcttcccaggaaaaaggacaggcagaaagagtttgagacctgaatagctcccagatttcagtcttttaatgtttttgttaactttgggttaaaaaaaaaaaaagtctgattggttttaattgaaggaaagatttgtactacagttcttttgttgtaaagagttgtgttgttcttttcccccaaagtggtttcagcaatatttaaggagatgtaagagctttacaaaaagacacttgatacttgttttcaaaccagtatacaagataagcttccaggctgcatagaaggaggagagggaaaatgttttgtaagaaaccaatcaagataaaggacagtgaagtaatccgtaccttgtgttttgttttgatttaataacataacaaataaccaacccttccctgaaaacctcacatgcatacatacacatatatacacacacaaagagagttaatcaactgaaagtgttccttcatttctgatatagaattgcaattttaacacacataaaggataaacttttagaaacttatcttacaaagtgtattttataaaattaaagaaaataaaattaagaatgttctcaatcaaacatcgtgtcctttgagtgaattgttctatttgacttcacaatagaaacttaataatcgtaccttctcaaga

SEQ ID NO: 9 - DNA, 17538 bp, Homo sapiens
atggaaatgttctgtatttgtgttgtctgatgagataaccactaactgtagtgctattgagcatttgaaacatggctagtgtaatcaatgaaccaaatttttaattttatttaattgtaattaattttaagtggccacatgcagggagtgactgctgcattggacagcacggctctaaattgagccttttttccttatttggtgaggcatacttgccttaagattgggaagtctatttttggaacctgctaccaatgctggtctcacacttgcaattctcagctgagccaagaggtgagagaaaggtcattttccattccaagatctcactctcccctgtgacactgaggaaactggcaagtgatgtgaaggctggagagcgtgtcctgtatgctggctctgtcccttctgcctgtgttgactgacatagttagttgctgcccttgctggtctcccttcctccaaccttgcctctctgagcacacctgacattcatctcatgacttccctaaaaacattctttgggaacaagaaactaacaaatcccaagtgacctatcacatatacaaacatacagggcagagtttggattcgcggtagaagaaagggaggttagacattaagaagaatggtctggtgatgacagttgtgagataatagaaacaggaaaaagaaatctaagttttctttctttttttaagaaccaataataatttctctcttttgactagtcagtagggctggggtggattggaggaagcttacatattccatgaacaagcctcttcctaaggtcctgtaagtgatcctgccccactgattagcccctagaagacccttcaaaggttggatctccaggagggagtgggggaggaaagccctgtaccaggcagcctctgctccattgctctgggggggtggggaagacaaaccctggtcatcccctcagtctgtagcccttttgtgtgagtgcctggcaagggtgacgtggggctgtttctgcgggcacagctgcagcaattaccggagtggaggcagggcccaggcagcactgccctccaagatcttcccttgggcttttcagcagtaaggggacatgcaccccaagggcctccacttggcctgaccttgctgcgggggctctctgtccccaggaacagtagagatggcaagcttatcgagaccctctctgcccagctgcctctgctccttcctcctcctcctcctcctccaagtgtcttccagctatgcaggtaagacatgttttttttcctgccctggggagaccctgaaaacagaaaggctagtttcctgggggttagctccttcaaacatcctcaagttggtatattatctttctaaaacatagacctactgacatgcctcccttcctcagaaaccttccgtgggtggttcttacagccttcaagatggagtccagactcttttttttttttgggacagagtctccctctgttgctcaggctggagtgcagtggcatgatctcggctcactgcaacctcagcctccctggttcaagcgattctcctgacttggcctcccaagtagcggagactacaggcgcctgccaccacacccagctaaatttgttcttttctttctttttttttttttttgggattttaggacagacggggtttcacatgttggccaggatggtctcgatctcttgacctgctgatccgcccgcctcagcttcccaaagtactgggattatgggcgtgagccactgcactaggcctaatttttttatttttagtagagatggggtttcaccatgttggccaggctggtctggaacccctgacctcaagtggtctgccctcctcagcctcccaaagttctgagattacaggcatgagccattgcgtctgacccagactccttaatgtgactaactccaggctttccttggactacttcttacttgtctttccagctttgtcttttcacctctccaattgagataaaataataacaacctcttggagttctcatcaggattacatgaaatgagatatgtaacatgcttagcagtgcctgtccatagtaaatctcaataaatgtttgtggaattataatatcttgtcatgtttgagactttgctctgcataatcaggcaccagtaggtttttataaaggaacccgtctgtcacgtgcagaggagaaataaacagaaagtttcccatcctcagggagccacctgactgacagaggcacagtgcatccactctccaggtctaggggagaaagcagccttatttcttagtagctcagaatctgacttgagaaacacatccacatagaaaaaaacaaggaactttttcgggtcagggtccgggacccacagtgaggtggaagatacaggggaaggaagagggaaatagagccatccccagggtggaagatctcagaagagaatttgggaaacaaggtatgaacaaggactgaatagtgagaagtgatggagagacagctaaagtagatggagtgtcaaaaccaaaacctctaagggtagaataggcagcaatttggccaagtcctaacagggaggcccataggaggattcaacctcaagatgctgtgccacattccaagagggaacctaaaggctgggctgaagagtcagagatggctacagctggcaaaaagatgggcagatgctgagaggagatgattgctaaaatgttctgtccaggacattcacagtatctctataaccagagtcttttttgtcgttgttgttctcaagaaggaaacttgaggccgggtgtggtggtttatgcccataatcccagcgctttggggccaaggcaggcggatcacctgaggtcaggagttcgagaccagcctggccaacagtgtgaaacctcatctttactaaaaatacaaaaattagctggatgcggcggtaggtgcctgtaatgccagctactcgggaggctgaggcaggagaatcacttgaacctgggaggcggaggttgcagggaggcggaggttgcagtgagccaagattgcaccactgcactccagcctgggcgacagagagtaagactgtctcaaaaaataaatgaataaataaaaaggaagaagaagaagaagaacaattgcaatcctccctggctctagaatgtcatttaaaagtcgagtgtcttcttccttccctgttttgaagcagcccttctcatgacaggcttgcttgccaaggttccctctgaccttaaatctcttccttttggtgtcttggacagggcagttcagagtgataggaccaagacaccctatccgggctctggtcggggatgaagtggaattgccatgtcgcatatctcctgggaagaacgctacaggcatggaggtggggtggtaccgcccccccttctctagggtggttcatctctacagaaatggcaaggaccaagatggagaccaggcacctgaatatcggggccggacagagctgctgaaagatgctattggtgagggaaaggtgactctcaggatccggaatgtaaggttctcagatgaaggaggtttcacctgcttcttccgagatcattcttaccaagaggaggcagcaatggaattgaaagtagaaggtgagtagtgccatataatattaggtattaactgttgggtggccaagaacaattattctctcaactgagatgagatccctcaacccaaacatctcagtcctgggaatgatttccataaaaatgtacacatcaataaacagaaactcatgcttagggatgtctgttgcatcattattcagagtagcaaggaaattgggatcaaaatcaatgcctttgagtaggtaagtgacagaatgaacaatggtagccatactgtgaatattatgcagggattaaaaagattattttagcactaggccagatggtttggggggctcctctaaggtattattgagtgataagagcaagctgctgtaggatacaaaaacaaaaacaaaaccctagggcatggtggtttgcctcgcagctactcaggaggctgagacgggaggctggcttgagcccaggggtttgcagttacagtgagctatgattgcaccactgcactccaacccgggtgacagagcaaagaccttcacccccactccctacccgtctctaaaaaaaacaaaaacaaaaacaaaaaaacccttgggcccagcgccgtggctcacgcctgtaatcccagcactgtgggaggccgaggtgggcagatcacaaggtcaggagatcgagaccatcctggctaaaacggtgaaaccccgtctctactaaaaatacaaaaaaaaaaaaaaaatttagccaggcatggtagcaggcgcctgtagtcccagctactcgggaggctgaggcaggagaatggcgtgaacccggaagcggaggttgcagtgagccaaaatccttccactgcactccagcatgggggacacagcgagactccgtctcaaaaaaaaaaaaaaaaccctgtatttgtgagcgcacacacacacacacacacacacacacctgtgcttggtcctagtgaataagcaagtaaatcaaatgtctaaatataattatagaaaggagatgtcaccttttggctgtacctccactatttcattctgcagaattgcagaatttcttttttttttcctttctttcttttctttttttttttgacacagagtctcgctctgtaacccaggctggagtgcaatggcgccctccgcctcctgggttcaagtgattctcctgcctcagcctcccgagtagctgggattacaggtgcccaccaccacacccagctaatttttgtatttttagtagagacagggtttcaccaggttgtcaaggttggtctcaaactcctgacctcaggtgatccactcgcctcagactcccaaagtgctgggattacaggcatgagccatggtgcccggcctcagaatttcattttcaacatgttttgcatgatgggtgattttggagaatattttttgctctatcgcaggatgattaagatgtggacaaggtgaagccgatggagggggagctttgaaagttacttgctatttaattgaggaactaaactgctttgagagcctgggggtcagatcctctgccttttcctcctccccacctgcagtgcaaacatcagacaattgatcactattgtatcttggaggtgggagtgaccattgcagtgctgggaccagaagatggcattgtatgtggaacaacaaagcactatttctagagactgcctgcagggatatggaaatagctttatgtgtctcagaatgttcttcatacagctgtttttattggggaaattctacttgccgaaaagtttgatagtgagaccctctccagtttgcagatttttctccttcctgctcaacaacttcctagctcagtaactgcctctcccaacaaactccctcagtttcaccacaccaaaaaaggaagacaagccggttgcggtggctcacacctataatcccaaaactttgggaggccgaggcgggtggatccacctgaggtcgggagttcgagactagcctgaccaacatggagaaaccctgtctctactaaaaacacaaaattagcctggcgtggtggcgcattcctgtaatcccagctgggaggctgaggcaggagaatcgcttgaaccccggaggcggaggttgcagtgagccaagatcgttccattacactccagtctgggcaagaaaagtggaactccatctccaaaaaaaaaaaaaaaaaaacaaggaagacaaaaagaaaagcagctaaagactttgcctcaggggagaaagttctcttttgggttgctatccacattccaacctcctgttcccacctcttcgtctgcatgcctaagaaactgttttacaagtaaataagggacgctttgtctaggctttggagccaggaagttgagacaaatttaggaatgagatgaagtaatggtattattgcaagtctcaggtgtaactacctctgctctttctctgaagagtttctaatttctcttgtttacttatttttttcttgtcatttttgggattttattactagttgtctctaatcctttctttaaattcttcattatgaaacataaaaacaaatgccaggcgcggcagctcacgcctgtaatcccagcactttgggaggccgaagcgggcagatcacccgggtcaggagttcgagaccagcctgatcaacatggagaaaccccgtctctactaaaaaatacaaaattagctaggcgtggtggcacatgccagtaatcccagctacttgagagactgaggcaggagaatcgcttgaaccgggaggcagaggttgcggtgagccaagatcgcgccattgcactccagcctgggcaacaagagcaaaactctgtctcaaaaaaaaaaaaccacatacaaaccagagataatattataatgagcctccaagtgcctaccaccttgctgcagcacttgtcaatccagggaccacccacctcaccggctccccactcattaccaccctcccctactcaattactgaggtaaatcctaggcagcatgatcatttcttttttttctttttatttattttgagacaggatctgtctctgtcacccaggctggagtgtagtggcatatctctgctcactgcagcctctgcctcccgggcagaagccatcctcccacctcagcctacatagtagctgggaccacaggcacacaccaccacacactgctaatgttttgtattttttgtagagactgggttttaccatgttgatcaggctggtctcaaactcctaggctcaagcaatcctcccacctcggcctcccaaagtgctagaattacaggcgcgagccactgcacccagcgaagaacactttttaaaaaataaataggccgggcgcggtggctcacacctgtaatcccagtactttgggagcccaaggagggcgaatcatgaggtcaagagattgagaccatcctaagtaacatggtgaaaccccatttctactacaaatacaaaaacaaaattagcctggcgtggtggcaggcgcctgtagtcccagctacttgggagctgaggcaggagaatggagtgaacccgggaggcggagcttgcagtgagctgagatcatgccactgcactcccccctggggcaacagagtgagactcccaaaaaaaaaaaaaaaagccccccctccccacacacaataatataaataaataaataaccacaatactattatcacatcttacaaactcaacaaaaatttcttaatatcatcaaatacccagtttgtgttcaaattttcctgattgtttcataaatatactcttacagttggtttcttttagcgagattcaaatgagacccacctgttgacctttgcccttagggtttcccagggtctgaattttgttgacgacattcccatgttgctatgtaatacggtcctccatgccctgtgtttttctgtaaactgatagatgtggaggtgcaatgacatttgtgtttgatttactttggcaaatatagttcatcagtgatactctatacttcttgttgctttacatccggaggctgataatgtctgcttttctctcttttctaattatttgtgaaaggaaaaatgtggggggttgggagaaaaaaacccttaagtacatactcgctaaatcacattgctacaggtaacttccattaagaacttgaaagtaaaggtagctgcattttcccctagggaacacaatgatagacaggagccttagtctacagcttgaaggattgtaattatacctaagcaaccctcctggaccagtttaatgttattagctgtgatgtatccctacctttgatgtcattatccttacttagctcccttaaagcagagatcaagatgaaaagggcttcagctgcagcatggcacatggagattagagtggggcttttggatgctgaggagcagacctagaatgggaaatagatgggagccacagaagtgaaggtccccctccctcattgctcaacctactccacatctccaggtctgcacatctgttcagttactgaatcctgtgtaagctaccttctttttcttttttcttttatttatttatttattttttttttgagatggagttttgctcttgttacccaggctggagtgcaatggtgcaatctcggctcactgcaccctccaactcccaggttcatgcaattctcctccctcagccttccaagtagctgggattacaggctgcaccaccatgtctggctaatttttgaaaaatcagtagagagagggtttcaccatgttggccaagccggtctcgaactcctgacctcaagtgatccacccaccttggcctcccaaaatgctgggattacaggtgtgagccaccatgcccgctgtaaactaccttcttaaaagctctagaagagggcttttaaccttttgttgtgtgtcatgcaccttccgcaagctgatgaagttgatagacccatctcagaattttttttttttttttgagacagtgtctcactctgtcacccaggattggttgcagtggcacgatcatgggtcattgcagcctccacctcccaggctcaagtgatcctcctgactcagcctcttgaatagctgagaccacaggcttgtgtcaccatgcccaggtaatttttaattttttttcgtagaggcagggtctcacattatgttgcccagtctggcctcgagaactcctgggctcaagcaatcttcctgccttgggctcccaaagtggtgggattacaggggagagccaccacacctagccaggaggatgttttaaatacaccaaataaaacatttatacccaaatacagttatccaaatattaaattaacaagagttagggtgaccctattaattagtgtaatttccaaatagtaatgaacataagtgatagtttgagatttctgtgacttttctaatgtgacgtgaaaatatttgtgatttttctttttcttttttttttttgagatggagtttcgctcttgttgcccaggctggagtgcaatggcaagatctcggctcacctcaacctccgcctcctgggttcaagcgattctcctgcctcagcctcttgagtagctgggattacaggactgtgccaccacgtccagctaattttgtatttttagtagaaacagggtttctccatgttggtcaggctggtcttgaactcccaacctcaggcgatccgcccgcctcggcctcccaaagtgctgggattacaggtgtgagccaccgcacctggccaatatttgtgatttttattgacgacaaagtcaaaggttctcttcatattattgtggtgtatcgcctacaagcataattaaaataaacactaaatttcagtttaaagtttactgaaaataaatatgtattttttattccctatttaagctttgaatcccctgacttcctataccattaccactgtcctagttcaggttcatgttgttttttactttaattgttatcacagtctcttaacatttctccctatgttctccagtcctgtaggtgctaaatctgacgtggtcacttctcagcttggaatccttcagtgcaccaccacagccttgaactacatatttgaaatacatatttattttcagtaaactttaaactgaaatttagtgtttattttaattatgcttgtaggcgatacaccacaataatatgaagagaacctttgactttgtcgtcaataaaaagtcccttgaggggacttcagatgtaagtcccttagctgctcgttaaaactcccccaggctgacccaatacacaatcttgactttaaaccacttgtcattctaaatcactagcatttcctggaaaaaaaagccatttttccttcagggctaagctcagggaccaattctgtgtcaccttctttgaatcctgatgatattcacttctttatttgacctgatttattgggccccagacaccatgctgagtgttggggattcagctctggacaatgtcaaatgtcagtcctgcctttcagatcctttctactgggtgagccctggagtgctggttctcctcgcggtgctgcctgtgctcctcctgcagatcactcttggcctcgtcttcctctgcctgcagtacagactgagaggtacagggcagagggtgggtggatcaggatcctttctttaaatgagctggcttcttggagctacaccacttaacatgtatttgtgagtgacttctgggttcagaagttcttctcactattgagtgataaagaaaaaaaataactccatgatgaaagagttttacatcttacggaatgctttcatatgaataatcggacctagcatttccctatgagctaactatgccatatagtaaccccattttacagaggatacaactgaggccaggagtagttcagtgacttactcaaaccgatataacttataagtggtagagctgaggcctctgtatcatacctagcagctccatgcaacttgggagagtgtgagcttcgaagtcagacaggtctaggctattaggagttttgaataaagatactgaagtgaaagtctctaccacacagtaggcgttcgaaaattgtttcctctttctccattcaacactgaggactcaggttcagctgctgatgaagctcctcttttttgcctagagctttcattctgagccttctcctcctaccaagtgtctccccaatgccagagcaggaagagtcttcactcctcccaatgccccacctcccatttgttactaagaggagaggagaaagtagcaaggagggtatggggaatgttctgggggaatgggtgttggtgcgatcaacaacaaagtcctttctctcaccttgaattcatcccagatgcctgcttgtttacttcttccacacaaaaaaaggccttcagccctcatggctgagcagaaagaatctgaatgttagagtcaggcagcctgggtttgaattccatctcaggtactgaactctatagcaaaattcttagattctccaagcttcagttgccttgtctgtcaaatagagaaaacatccttcgtcctaaattgtagggaggattaaagtcatgcaaagtgcctactacaaatccagtcacaaagtagctagctactcactaaatgttcagctcctccctcctcattcagatgggaagtggctttagataaacaaagtggcaacgcagtgggctggagcagctctgtgaactgagaatccaagaaaaggggcgaagagcagctgggatgtattggatgcttgtgctggcttggagcattgctcacattctttattcgctattgtatctagactatagctagagaaagagccgcaaccattggctttaaatccagtgctcttcctactctcctgaggttgtttccaggctgcagagaaatagcctgcacaaggggcccaggcgctgggtgtgggagggtccccaccgagagccagaacatgcaggaactaaaatgttgcctttttctattttaggaaaacttcgagcagagataggtgagttccagtcatcgtttctcccaattcttgccttttggttttttggcataacggaaatggtcccattcttggaccgtctctccctctcaataccctgttttcccctcagtttccctttctctacagtgggtgtgtcgtgcctagaacaagttttaagtaattaaataacaaagactcaggataaaaggatcctttttggagtgccctactaaatccatttccatttgtttctctttcagagaatctccaccggacttttggtaagttccggcatgtctaggccctcccaggtcaacttggtatttcactctagttccagtcacctgggggaacaaggacccctggctcctggttgagtcccttcctctcttctcttttctttctttaaataagaagtcatttgcatttaggattggtaaaatcataataaaaatactcatgtactgtttttatgtgccaggcactattctaactactttacaaaaacgttatcttattctgtttaactccttatgcacatgatctctcttttcaggaatgccaaaacagaggtaaatagatcgtttacacgtaaacctgatgtctggttggggaggtgaaacaaacagaaacaagacacaactgtatcacctgtacttatatttctgctttacaaactcaggatgtttccatgagtacagaacatgactaatcagagaagacctcatagaggaatagaaaagccaccaagccccactaggaattgacccctcaaggacatggtttctagcctttttgttcactgcagattgcccaatgcctaaagataatggcaacagaagagcacccaaatatttgttagataaatgttgcagacactagaaggtgtcattagggcacagatggtaccttctctgagcaaacttccttcacagctcctcctcccgaggctgtaggtgactctactcttgtcacctggcacacagagttctatcgtacgatttaggaaattagaccagtgtgtggaccacacacacacacatctttacacacccaaagaggaggaatagtatctttgttttggaggacttgactatgaaaggtcttaactcctttttgtaccatgaatctctctggcactccagtgaagtctaaaggacccctttgcagaatgtttttaaatatacacataaaatagaacacataggattgcaaaaacaatcattgtactaaaatacagttatcaaccgataatcacatttgtgatatagtaacataaatgtttcttttttttttttttggaggcagagtttggctcttgtcacccaggctggagtgcaatggcgcgatctaggctcactgaaacctctgcctcccgggttcaagcgattctcagcctcctgagtagctgggattacaggtgcccgccaccacacccagctaatttttgtatttttagtagagactaggtttcaccaggttggccaggctggcctcgaactcctgacctcaggtgatccacctgccttggcctcccaaagtgctgggattacgggcatgagccaccgtgcccggccataaatatttctttagccaaagtaatacattaagtaatgtagcagcaagtctaataacctgtaatttctttctttctttctttctttctttttttttgagatgaagtttttttgagatggagtgcaatggcacaatctcggctcactgcaacctccacctcctgggttcaagcgattctcctgcctcagcctcccaagttgctggaactacaggcgcatgccaccatgcccagctaatttttgtatttttagtagagacggggtttcaccatgttggccaggctggtcttgaacccctgacctcaggtgatctgcctgccttggccttccaaagtgctgggattacaggcatgagccaccaggcccagcccaataacctttaatttcaacatactaataaacataaacagtatttcaagatttctgcaataactctaatgggaatgaaaacatctgtggcttccattggtaattaagtcacaggtactgctcatattgtggttagttgtaaaatgttttggtttgttttgttttttccaagacttgggggaatgggtgttggtgggatcaacaagagtcttgctctgtggcccaggctggagtgcaggggcaggatcttggctcactgcaacctccgcctcccaggttcaagcgattctcctgcctcagcctcctgagtagctggcattacaggcatgtgccaccacgcccagctaatttttacatttttagtagagatggggtttcaccatgttggcctggctggtcttgaactcttggcctcatgatccacccgtctcggactcccagagtgttgggattacaggcatgagccaccacacctggcagttgttacatttttaatgaaagaaaatgttaaatccagttattgaaaataaggaggcagtacttttctcatccaagttcatggactttctgaattttgtccccagagtcctttggtgttctaggaccccaggttaaggaacccaaaaagacaggtgggtggggcatgagggggaacacatgttaatccctgtttgttctggtgaacaattcagatccccactttctgagggtgccctgctggaagataaccctgtttgtaattgtgccggttcttggacccttggttgccttgatcatctgctacaactggctacatcgaagactagcaggtgcagtggctgggcagcaggcaagaccaccaaatagtgggggaccaagtcagctctgaatgggaagccaaaagagaatagaaccaggactcaagattaggggagctgggatttccttattcctctgtccccatgcccaaccccaggctcttctgagaaactgtgaagagaaccacttactggatctgtgggatcccccagtggaaagggcagtgtgggtcactccaaatgtccatagggaggatgtggggaaggtgctattcatcttccactaatcacatatttgtttctttttgttttcagggcaattccttgaagagctacgtaagttctcttctctctgttataagcagagaataaaaagccaggaaagggagacagaagcaacaagaggaagaggcgggctattgagggatcacattcccagaggaaaggaggagctggagagcctgggtggagggaagactcctcctgggaggtagagggcaaagaagccagctgttagagacacatttacaggtggcagagaagctggaggcactcctatctgccacctgatccattcctccttcactgcccctaagcaggaatccaaccctagctggtctcattgcccattccacagcaactgcccagtgcctcacctctcagatcaaccattgaggcaggaatggagacaagatgaccccaagggcttttcttctccctagttcaatggttttatgatacaaactactgacatacgtttttcaagttattttctccttcttctaggaaatcccttctgagtgatgtcacatcttggcaggggtggaggagagcctggttgcccagggatttgtccttggggacatctcatccatcaagttgcacactcactggcatctttgctatggggacattccaatttgcactttcaggaacactctgaattccaagtagaattgatttcccttcttctgtcatctaccttttctcttcattttcccatttttattacccttctttccatttctctctccagtcttccacctggaagccctctctggctaaggacaggcaggtgcccctctctccatcagaggacacctgtactggagagcaacacaggatggtctctgccatgaactggaggccaggaatctcctcactgaaaattacagtatggtaactttgcaaatggtggttgtttcttccaagactccagccctgattgcgcaaaactgaaaggcatgtgaagggaaggaagaggaagagtgcaaaacattgaagagagagctgagtgagctgaagagtgaggatatgagtagccccaacccaaacctggagatggggagaaacctacagaatactagccagagctcctccttgtcttggcagcctactagggacctggggaagcaaaaacgaaagctgggcaacatgcctgctttagaatgttttccttctacttacacatcttccacaggtctcagaatctttccttcctctcatccttttctcctatctacatatctatcagagtatccactgtttattcaacaactactacttgatggtcagacacaaacaaacaagctaggtgctaattaataaagatacgagttttggccgggtgcggtggctcacgcctgtaatcccagcactttgggaggccgaggcgggcgaatcacgaggtcaggagttcaagaccagcctggccaacatggtgaaaccccatctctactaaaaatacaaacaattaactgagcatagtggtgggcacctataataccagctactccggaggctgaggcaggagaatcgcttgaacccaggaggcagaggttgcagtgagctgagatcgcgccactgcactctagccggagtgacagagtaagactctgtctcaaaaataaataaataaataaataaataaataaataaataaataaaaaataataatacaagttttcataagcacacttctaaccccttgtcttttatgtatttccttccttatccacgcacctgtctccctctactccagcctcattaccccagaggtcagtcctcaggaaaactaaacacaaagaaagagctcagtcagaaaggccatttatttatgtttcaagatgctcactgcctcctttgttttgtctcctttgcaggccttctctcttaggcctcttctcctgggggtatggatcctggggggagattgatcacctccatgcttccattcctccccagccatagtggggacatcatgagagaagccaagccactggcccaggatcacccggcatttatggtggctgctctggcacaggtccttgcctttatagcccctccagtgatccataaggccctctttctccccaaaggagaggtcacagatagggcaaaggtagctcttctgcttccagtgggtctgctggtgtctgaccagcctggaaaatgagctgaaagacttgctgcaatggaagcagtagttgggcggctctgtgaggtggcccttctggtgtctggagagataggatttcttgctaaaagtcaaagaacaatgggggcaacagaagacattgagtcttgagggcttcactggatgagagttggatctggcatcctgacagagggttccagtgatgggtgcctgggtcctggtcacaggtgcttggttcttaagtacagatgcctggttctgggccataggaccctcagttctaaatatgggttcctgggacctggccactggtgcatggttcacatccaaaagcccctggatggacctctggcttctggcgatgggtgtctggaattcagcctgggtgcctggaatcctcaaagtacactcctggtttccatccactggctcctggttttggtgtatcttctggtggcgtttgagctcagactggtcccggaagctcttcccacacacagagcatgaatggggccggtaacccagatggacgcggcggtgacgacttagtccagaagcatcacagtaggtcttgtcacagagcgtgcaacagaagggcctctccccaagatgcatgcgtctgtgatagctgagggacttggggctccgaaacaacttcccacactgactgcagctgttagtcagcttgggattgtgaacaaactggtggctatagaggtaggagcgcctgctgaaacatttggcacaggtgtagcaaaa

SEQ ID NO: 10 - DNA, 327 bp, Rattus norvegicus
tttgtatgtcattgcaggattcatgctttccagtgtgtcatctatggaactgcctctttcttcttcctttatggggccctcctgctggctgagggcttctacaccaccggcgctgtcaggcagatctttggcgactacaagaccaccatctgcggcaagggcctgagcgcaacggtaacagggggccagaaggggaggggttacagaggccaacatcaagctcattctttggagcgggtgtgtcattgtttgggaaaatggctaggacatcccgacaaggtgatcatcctcaggattttgtggcaataacaaggggtggggggacaa

SEQ ID NO: 11 - DNA, 2013 bp, Rattus norvegicus
ctgtatcagtgctcctcgtcgcctcactgtacttcacggaagagacttggttgactggccacttggagcggaatcaggagacattcccaactcagagagactgagccctagctcgcccacttgctggacaagatgatattccttaccaccctgcctctgttttggataatgatttcagcttctcgaggggggcactggggtgcctggatgccctcgtccatctcagccttcgagggcacgtgtgtctccatcccctgccgtttcgacttcccggatgagctcagaccggctgtggtacatggcgtctggtatttcaacagtccctaccccaagaactacccgccagtggtcttcaagtcccgcacacaagtggtccacgagagcttccagggccgtagccgcctgttgggagacctgggcctacgaaactgcaccctgcttctcagcacgctgagccctgagctgggagggaaatactatttccgaggtgacctgggcggctacaaccagtacaccttctcggagcacagcgtcctggacatcatcaacacccccaacatcgtggtgcccccagaagtggtggcaggaacggaagtagaggtcagctgcatggtgccggacaactgcccagagctgcgccctgagctgagctggctgggccacgaggggctaggggagcccactgttctgggtcggctgcgggaggatgaaggcacctgggtgcaggtgtcactgctacacttcgtgcctactagagaggccaacggccaccgtctgggctgtcaggctgccttccccaacaccaccttgcagttcgagggttacgccagtctggacgtcaagtaccccccggtgattgtggagatgaattcctctgtggaggccattgagggctcccacgtcagcctgctctgtggggctgacagcaacccgccaccgctgctgacttggatgcgggatgggatggtgttgagggaggcagttgctgagagcctgtacctggatctggaggaggtgaccccagcagaggacggcatctatgcttgcctggcagagaatgcctatggccaggacaaccgcacggtggagctgagcgtcatgtatgcaccttggaagcccacagtgaatgggacggtggtggcggtagagggggagacagtctccatcctgtgttccacacagagcaacccggaccctattctcaccatcttcaaggagaagcagatcctggccacggtcatctatgagagtcagctgcagctggaactccctgcagtgacgcccgaggacgatggggagtactggtgtgtagctgagaaccagtatggccagagagccaccgccttcaacctgtctgtggagtttgctcccataatccttctggaatcgcactgtgcagcggccagagacaccgtgcagtgcctgtgtgtggtaaaatccaacccggaaccctccgtggcctttgagctgccttcccgcaacgtgactgtgaacgagacagagagggagtttgtgtactcagagcgcagcggcctcctgctcaccagcatcctcacgctccggggtcaggcccaagccccaccccgcgtcatttgtacctccaggaacctctacggcacccagagcctcgagctgcctttccagggagcacaccgactgatgtgggccaaaatcggccctgtgggtgctgtggtcgcctttgccatcctgattgccattgtctgctacatcacccagacaagaagaaaaaagaacgtcacagagagccccagcttctcagcgggagacaaccctcatgtcctgtacagccccgaattccgaatctctggagcacctgataagtatgagagtgagaagcgcctggggtccgagaggaggctgctgggccttaggggggaacccccagaactggacctcagttattcccactcagacctggggaaacgacccaccaaggacagctacaccctgacagaggagctggctgagtacgcagaaatccgagtcaagtga

SEQ ID NO: 12 - protein, 171 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 13 - protein, 274 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 14 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITLGLVFLCLQYRLRGKLRAEIENLHRTFDPHFLRVPCWKITLFVIVPVLGPLVALIICYNWLHRRLAGQFLEELRNPF

SEQ ID NO: 15 - protein, 18 aa, Rattus norvegicus
APKRGSGKDSHTRTTHYG

SEQ ID NO: 16 - protein, 23 aa, Homo sapiens
VLGGGCALLRCPALDSLTPANED

(19) United States
(12) Patent Application Publication
(10) Pub. No.: US 2003/0108528 A1
(43) Pub. Date: Jun. 12, 2003

(54) ACTIVATED T-CELLS, NERVOUS SYSTEM-SPECIFIC ANTIGENS AND THEIR USES

(76) Inventors: MICHAL EISENBACH-SCHWARTZ, REHOVOT (IL); IRUN R. COHEN, REHOVOT (IL); GILA MOALEM, REHOVOT (IL); PIERRE BESERMAN, MOSHAV SITRIYA (IL); ALON MONSONEGO, REHOVOT (IL)

Correspondence Address: BROWDY AND NEIMARK, P.L.L.C., 624 NINTH STREET, NW, SUITE 300, WASHINGTON, DC, 20001-5303, US

(21) Appl. No.: 09/218,277

(22) Filed: Dec. 22, 1998

Related U.S. Application Data
(63) Continuation-in-part of application No. PCT/US98/14715, filed on Jul. 21, 1998.

(30) Foreign Application Priority Data
May 19, 1998 (IL) IL 124550

(51) Int. Cl. A61K 48/00; A61K 38/17; A61K 45/00; A61K 39/385

(52) U.S. Cl. 424/93.7; 424/185.1; 514/44

(57) ABSTRACT

The present invention discloses compositions and methods for the treatment of injury or disease of the nervous system CNS. In a particular embodiment, the invention provides methods of treatment using non-recombinant activated antiself T-cells that recognize an antigen of the NS or a peptide derived therefrom or a derivative thereof to promote nerve regeneration or to prevent or inhibit axonal degeneration within the NS. The invention also provides methods of treatment using a NS-specific antigen or peptide derived therefrom or a derivative thereof or a nucleotide sequence encoding said antigen or peptide to promote nerve regeneration or to prevent or inhibit axonal degeneration in NS, i.e., the CNS and/or PNS. The NS-specific antiself activated T-cells may be administered alone or in combination with NS-specific antigen or peptide derived therefrom or a derivative thereof or a nucleotide sequence encoding said antigen or peptide or any combination thereof.

The present application is a continuation-in-part of PCT/US98/14715, filed Jul. 21, 1998. The present application claims priority benefit under 35 U.S.C. §119 of copending Israeli patent application IL 124550, filed May 19, 1998, the disclosure of which is incorporated herein by reference in its entirety and priority benefit under 35 U.S.C. §120 of PCT/US98/14715, filed Jul. 21, 1998.

### 1. FIELD OF THE INVENTION

The present invention relates to compositions and methods for the promotion of nerve regeneration or prevention or inhibition of axon degeneration to ameliorate the effects of injury or disease of the nervous system (NS). In certain embodiments, activated antiself T-cells, a NS-specific antigen or peptide derived therefrom or a nucleotide sequence encoding a NS-specific antigen or peptide derived therefrom are/is used to promote nerve regeneration or to prevent or inhibit axonal degeneration caused by injury or disease of nerves within the CNS or PNS of a human subject. The compositions of the present invention may be administered alone or may be optionally administered in any desired combination.

### 2. BACKGROUND OF THE INVENTION

The nervous system comprises the central and the peripheral nervous system (PNS). The central nervous system (CNS) is composed of the brain and spinal cord; the PNS consists of all the other neural elements, namely the nerves and ganglia outside the brain and spinal cord.

Damage to the NS may result from a traumatic injury, such as penetrating trauma or blunt trauma, or a disease or disorder, including but not limited to Alzheimer's disease, Parkinson's disease, multiple sclerosis, Huntington's disease, amyotrophic lateral sclerosis (ALS), Diabetic neuropathy, senile dementia, and ischemia.

Maintenance of CNS integrity is a complex ‘balancing act’ in which compromises are struck with the immune system. In most tissues, the immune system plays an essential part in protection, repair and healing. In the CNS, because of its unique immune privilege, immunological reactions are relatively limited (Streilein, J. W., 1993, Curr. Opin. Immunol. 5:428-432; Streilein, J. W., 1993, Science, 270:1158-1159). A growing body of evidence indicates that the failure of the mammalian CNS to achieve functional recovery after injury is a reflection of an ineffective ‘dialog’ between the damaged tissue and the immune system. For example, the restricted communication between the CNS and blood-borne macrophages affects the capacity of axotomized axons to regrow; transplantation of activated macrophages can promote CNS regrowth (Lazarov Spiegler, O., et al., 1996, FASEB J., 10:1296-1302; Rapalino, O. et al., 1998, Nature Med. 4:814-821).

Activated T cells have been shown to enter the CNS parenchyma, irrespective of their antigen specificity, but only T cells capable of reacting with a CNS antigen seem to persist there (Hickey, W. F., et al., 1991, J. Neurosci. Res. 28:254-260). T cells reactive to antigens of CNS white matter, such as myelin basic protein (MBP), can induce the paralytic disease experimental autoimmune encephalomyelitis (EAE) within several days of their inoculation into naive recipient rats (Ben Nun, A., et al., 1981, Eur. J. Immunol. 11:195-199). Anti-MBP T cells may also be involved in the human disease multiple sclerosis (Ota, K., et al., 1990, Nature 346:183-187; Martin, R., 1997, J. Neural Transm. Suppl. 49:53-67). However, despite their pathogenic potential, anti-MBP T-cell clones are present in the immune systems of healthy subjects (Burns, J., et al., 1983, Cell. Immunol. 81:435-440; Pette, M., et al., 1990, Proc. Natl. Acad. Sci. USA 87:7968-7972; Martin, R., et al., 1990, J. Immunol. 145:540-548; Schiuesener, H. J., et al., 1985, J. Immunol. 135:3128-3133). Activated T cells, which normally patrol the intact CNS, transiently accumulate at sites of CNS white matter lesions (Hirschberg, D. L., et al., 1998, J. Neuroimmunol. 89:88-96).

A catastrophic consequence of CNS injury is that the primary damage is often compounded by the gradual secondary loss of adjacent neurons that apparently were undamaged, or only marginally damaged, by the initial injury (Faden, A. I., et al., 1992, Trends Pharmacol. Sci. 13:29-35; Faden, A. I., 1993, Crit. Rev. Neurobiol. 7:175-186; McIntosh, T. K., 1993, J. Neurotrauma 10:215-261). The primary lesion causes changes in extracellular ion concentrations, elevation of amounts of free radicals, release of neurotransmitters, depletion of growth factors, and local inflammation. These changes trigger a cascade of destructive events in the adjacent neurons that initially escaped the primary injury (Lynch, D. R., et al., 1994, Curr. Opin. Neurol. 7:510-516; Bazan, N. G., et al., 1995, J. Neurotrauma 12:791-814; Wu, D., et al., 1994, J. Neurochem. 62:37-44). This secondary damage is mediated by activation of voltage-dependent or agonist-gated channels, ion leaks, activation of calcium-dependent enzymes such as proteases, lipases and nucleases, mitochondrial dysfunction and energy depletion, culminating in neuronal cell death (Yoshina, A., et al., 1991, Brain Res. 561:106-119; Hovda, D. A., et al., 1991, Brain Res. 567:1-10; Zivin, J. A., et al., 1991, Sci. Am. 265:56-63; Yoles, E., et al., 1992, Invest. Ophthalmol. Vis. Sci. 33:3586-3591). The widespread loss of neurons beyond the loss caused directly by the primary injury has been called ‘secondary degeneration’.

Another tragic consequence of CNS injury is that neurons in mammalian CNS do not undergo spontaneous regeneration following an injury. Thus, a CNS injury causes permanent impairment of motor and sensory functions.

Citation or identification of any reference in this section or any other part of this specification shall not be construed as an admission that such reference is available as prior art to the present invention.

### 3. SUMMARY OF THE INVENTION

The present invention is directed to methods and compositions for the promotion of nerve regeneration or prevention or inhibition of axonal degeneration to ameliorate the effects of injury or disease of the nervous system (NS). The present invention is based, in part, on the Applicants' unexpected discovery, that non-recombinant antiself T-cells that recognize an antigen of the NS or a peptide derived therefrom promote nerve regeneration or confer neuroprotection. As used herein, “neuroprotection” refers to the prevention or inhibition of degenerative effects of injury or disease in the NS. Until recently, it was thought that the immune system excluded immune cells from participating in nervous system repair. It was quite surprising to discover that non-recombinant NS-specific antiself activated T-cells can be used to promote nerve regeneration or to protect nervous system tissue from secondary degeneration which may follow damage caused by injury or disease of the CNS or PNS, in particular, a lesion other than a neoplasm or an autoimmune disease affecting the NS.

“Activated T-cell” as used herein includes (i) T-cells that have been activated by exposure to a cognate antigen or peptide derived therefrom or derivative thereof and (ii) progeny of such activated T-cells. As used herein, a cognate antigen is an antigen that is specifically recognized by the T-cell antigen receptor of a T-cell that has been previously exposed to the antigen.

In an embodiment, the present invention provides pharmaceutical compositions comprising a therapeutically effective amount of non-recombinant, NS-specific antiself activated T-cells and methods of use of such compositions for promotion of nerve regeneration or for prevention or inhibition of axonal degeneration in the CNS or PNS in which the amount is effective to ameliorate the effects of an injury or disease of the NS. “INS-specific antiself activated T-cell” as used herein refers to an activated T-cell having specificity for an antigen of the NS or a peptide derived therefrom. Preferably, the NS-specific antiself activated T cells are used to promote nerve regeneration or to prevent or inhibit the effects of disease in which the disease is not an autoimmune disease or a neoplasm.

The present invention also provides pharmaceutical compositions comprising a therapeutically effective amount of a NS-specific antigen or peptide derived therefrom or derivative thereof and methods of use of such compositions for promotion of nerve regeneration or for prevention or inhibition of axonal degeneration in the CNS or PNS in which the amount is effective to activate T-cells in vivo or in vitro wherein the activated T-cells inhibit or ameliorate the effects of an injury or disease of the NS. “INS-specific antigen” as used herein refers to an antigen that specifically activates T-cells such that following activation the activated T-cells accumulate at a site of injury or disease in the NS. Preferably, the NS-specific antigen is used to promote regeneration or to prevent or inhibit the effects of disease in which the disease is not an autoimmune disease or a neoplasm. In an embodiment, the peptide derived from a NS-specific antigen is a “cryptic epitope” of the antigen. A cryptic epitope activates specific T cells after an animal is immunized with the particular peptide, but not with the whole antigen. In another embodiment, the peptide derived from a NS-specific antigen is an immunogenic epitope of the antigen. “Derivatives” of NS-specific antigens or peptides derived therefrom as used herein refers to analogs or chemical derivatives of such antigens or peptides as described below, see Section 5.2.

The present invention also provides pharmaceutical compositions comprising a therapeutically effective amount of a nucleotide sequence encoding a NS-specific antigen or peptide derived therefrom or derivative thereof and methods of use of such compositions for promotion of nerve regeneration or for prevention or inhibition of axonal degeneration in the CNS or PNS in which the amount is effective to ameliorate the effects of an injury or disease of the NS.

In the practice of the invention, therapy for amelioration of effects of injury or disease comprising administration of NS-specific antiself activated T-cells may optionally be in combination with a NS-specific antigen or peptide derived therefrom or derivative thereof or a nucleotide sequence encoding a NS-specific antigen or peptide derived therefrom.

### 4. BRIEF DESCRIPTION OF THE FIGURES

FIG. 1 shows T-cell presence in injured optic nerve 1 week after injury. Adult Lewis rats were injected with activated T cells of the anti-MBP (T_{MBP}), anti-OVA (T_{OVA}), or anti-p277 (Tₚ₂₇₇) lines, or with PBS, immediately after unilateral crush injury of the optic nerve. Seven days later, both the injured and uninjured optic nerves were removed, cryosectioned and analyzed immunohistochemically for the presence of immunolabeled T cells. T cells were counted at the site of injury and at randomly selected areas in the uninjured optic nerves. The histogram shows the mean number of T cells per mm²±s.e.m., counted in two to three sections of each nerve. Each group contained three to four rats. The number of T cells was considerably higher in injured nerves of rats injected with anti-MBP, anti-OVA or anti-p277 T cells; statistical analysis (one-way ANOVA) showed significant differences between T cell numbers in injured optic nerves of rats injected with anti-MBP, anti-OVA, or anti-p277 T cells and in injured optic nerves of rats injected with PBS (P<0.001); and between injured optic nerves and uninjured optic nerves of rats injected with anti-MBP, anti-OVA, or anti-p277 T cells (P<0.001).

FIG. 2 illustrates that T cells specific to MBP, but not to OVA or p277 or hsp60, protect neurons from secondary degeneration. Immediately after optic nerve injury, rats were injected with anti-MBP, anti-OVA or anti-p277 T cells, or with PBS. The neurotracer dye 4-Di-10-Asp was applied to optic nerves distal to the site of the injury, immediately after injury (for assessment of primary damage) or 2 weeks later (for assessment of secondary degeneration). Five days after dye application, the retinas were excised and flat-mounted. Labeled retinal ganglion cells (RGCs) from three to five randomly selected fields in each retina (all located at approximately the same distance from the optic disk) were counted by fluorescence microscopy. RGC survival in each group of injured nerves was expressed as the percentage of the total number of neurons spared after the primary injury (42% of axons remained undamaged after the primary injury). The neuroprotective effect of anti-MBP T cells compared with that of PBS was significant (P<0.001, one-way ANOVA). Anti-OVA T cells or anti-p277 T cells did not differ significantly from PBS in their effects on the protection of neurons that had escaped primary injury (P>0.05, one-way ANOVA). The results are a summary of five experiments. Each group contained five to ten rats.

FIGS. 3(A-C) present photomicrographs of retrogradely labeled retinas of injured optic nerves of rats. Immediately after unilateral crush injury of their optic nerves, rats were injected with PBS (FIG. 3A) or with activated anti-p277 T cells (FIG. 3B) or activated anti-MBP T cells (FIG. 3C). Two weeks later, the neurotracer dye 4-Di-10-Asp was applied to the optic nerves, distal to the site of injury. After 5 days, the retinas were excised and flat-mounted. Labeled (surviving) RCGs, located at approximately the same distance from the optic disk in each retina, were photographed.

FIGS. 4(A-B) show that clinical severity of EAE is not influenced by an optic nerve crush injury. For the results presented in FIG. 4A, Lewis rats, either uninjured (dash line) or immediately after optic nerve crush injury (solid line), were injected with activated anti-MBP T cells. EAE was evaluated according to a neurological paralysis scale. [Data points represent ±s.e.m.] These results represent a summary of three experiments. Each group contained five to nine rats. FIG. 4B shows that the number of RGCs in the uninjured optic nerve is not influenced by injection of anti-MBP T cells. Two weeks after the injection of anti-MBP T cells or PBS, 4-Di-10-Asp was applied to the optic nerves. After 5 days the retinas were excised and flat-mounted. Labeled RGCs from five fields (located at approximately the same distance from the optic disk) in each retina were counted and the average number per mm² was calculated. There was no difference between the numbers of labeled RGCs in rats injected with anti-MBP T cells (T_{MBP}) and in PBS-injected control rats.

FIG. 5 shows that T cells specific to p51-70 of MBP protect neurons from secondary degeneration. Immediately after optic nerve injury, rats were injected with anti-MBP T cells, anti-p51-70 T cells, or PBS. The neurotracer dye 4-Di-10-Asp was applied to optic nerves distal to the site of the injury, immediately after injury (for assessment of primary damage) or 2 weeks later (for assessment of secondary degeneration). Five days after dye application, the retinas were excised and flat-mounted. Labeled retinal ganglion cells (RGCs) from three to five randomly selected fields in each retina (all located at approximately the same distance from the optic disk) were counted by fluorescence microscopy. RGC survival in each group of injured nerves was expressed as the percentage of the total number of neurons spared after primary injury. Compared with that of PBS treatment, the neuroprotective effects of anti-MBP and anti-p51-70 T cells were significant (P<0.001, one-way ANOVA).

FIGS. 6(A-B) show that anti-MBP T cells increase the compound action potential (CAP) amplitudes of injured optic nerves. Immediately after optic nerve injury, rats were injected with either PBS or activated anti-MBP T cells (T_{MBP}). Two weeks later, the CAPs of injured (FIG. 6A) and uninjured (FIG. 6B) nerves were recorded. There were no significant differences in mean CAP amplitudes between uninjured nerves obtained from PBS-injected and T cell-injected rats (n=8; p=0.8, Student's t-test). The neuroprotective effect of anti-MBP T cells (relative to PBS) on the injured nerve on day 14 after injury was significant (n=8; p=0.009, Student's t-test).

FIG. 7 illustrates inhibition of secondary degeneration after optic nerve crush injury in adult rats. See text, Section 8, for experimental details. Rats were injected intradermally through the footpads with a 21-mer peptide based on amino acid residues 35-55 (MOG p35-55) of myelin/oligodendrocyte glycoprotein (chemically synthesized at the Weizmann Institute, Israel) (50 μg/animal) or PBS ten days prior to optic nerve crush injury or MOG p35-55 in the absence of crush injury. MOG p35-55 was administered with Incomplete Freund's Adjuvant. Surviving optic nerve fibers were monitored by retrograde labeling of retinal ganglion cells (RGCs). The number of RGCs in rats injected with PBS or MOG p35-55 was expressed as a percentage of the total number of neurons in rats injected with MOG p35-55 in the absence of crush injury.

FIG. 8 illustrates inhibition in adult rats of secondary degeneration after optic nerve crush injury by MBP. See text, Section 9, for experimental details. MBP (Sigma, Israel) (1 mg in 0.5 ml saline) was administered orally to adult rats by gavage using a blunt needle. MBP was administered 5 times, i.e., every third day beginning 2 weeks prior to optic nerve crush injury. Surviving optic nerve fibers were monitored by retrograde labeling of retinal ganglion cells (RGCs). The number of RGCs in treated rats was expressed as a percentage of the total number of neurons in untreated rats following the injury.

FIG. 9 shows the nucleotide sequence of rat myelin basic protein gene, SEQ ID NO: ______, Genbank accession number M25889 (Schaich et al., 1986, Biol. Chem. 367, 825-834).

FIG. 10 shows the nucleotide sequence of human myelin basic protein gene, SEQ ID NO: ______, Genbank accession number M13577 (Kamholz et al., 1986, Proc. Natl. Acad. Sci. U.S.A. 83 (13), 4962-4966).

FIGS. 11(A-F) show the nucleotide sequences of human myelin proteolipid protein gene exons 1-7, SEQ ID NO: ______, Genbank accession numbers M15026-M15032 respectively (Diehl et al., [published erratum appears in Proc Natl Acad Sci USA, 1991, 86(6):617-8] Proc. Natl Acad. Sci. U.S.A. 83 (24), 9807-9811 (1986)).

FIG. 12 shows the nucleotide sequence of human myelin oligodendrocyte glycoprotein gene, SEQ ID NO: ______, Genbank accession number Z48051 (Roth et al., submitted (Jan. 17, 1995) Roth, CNRS UPR 8291, CIGH, CHU Purpan, Toulouse, France, 31300; Gonzalez et al., 1996, Mol. Phylogenet. Evol. 6, 63-71).

FIG. 13 shows the nucleotide sequence of rat proteolipid protein and variant, SEQ ID NO: ______, Genbank accession number M16471 (Nave et al., 1987, Proc. Natl. Acad. Sci. U.S.A. 84, 5665-5669).

FIG. 14 shows the nucleotide sequence of rat myelin-associated glycoprotein, SEQ ID NO: ______, Genbank accession number M14871 (Arquint et al., 1987, Proc. Natl Acad. Sci. U.S.A. 84, 600-604).

FIG. 15 shows the amino acid sequence of human myelin basic protein, SEQ ID NO: ______, Genbank accession number 307160 (Kamholz et al., 1986, Proc. Natl. Acad. Sci. U.S.A. 83 (13), 4962-4966).

FIG. 16 shows the amino acid sequence of human proteolipid protein, SEQ ID NO: ______, Genbank accession number 387028.

FIG. 17 shows the amino acid sequence of human myelin oligodendrocyte glycoprotein, SEQ ID NO: ______, Genbank accession number 793839 (Roth et al., 1995, Genomics 28 (2), 241-250; Roth Submitted (Jan. 17, 1995) Roth CNRS UPR 8291, CIGH, CHU Purpan, Toulouse, France, 31300; Gonzalez et al., 1996, Mol. Phylogenet. Evol. 6, 63-71).

### 5. DETAILED DESCRIPTION OF THE INVENTION

Merely for ease of explanation, the detailed description of the present invention is divided into the following sub-sections (1) non-recombinant, NS-specific antiself activated T-cells; (2) NS-specific antigens, peptides derived therefrom and derivatives thereof; (3) nucleotide sequences encoding NS-specific antigens and peptides derived therefrom; (4) therapeutic uses of non-recombinant, NS-specific antiself activated T-cells, NS-specific antigens, peptides derived therefrom and derivatives thereof, and nucleotide sequences encoding NS-specific antigens and peptides derived therefrom; and (5) formulations and modes of administration of non-recombinant, NS-specific antiself activated T-cells, NS-specific antigens, peptides derived therefrom and derivatives thereof, and nucleotide sequences encoding NS-specific antigens and peptides derived therefrom.

### 5.1 NS-Specific Antiself Activated T-Cells

NS-specific antiself activated T-cells (ATCs) can be used for ameliorating or inhibiting the effects of injury or disease of the CNS or PNS that result in NS degeneration or for promoting regeneration in the NS, in particular the CNS.

The NS-specific activated T-cells are preferably autologous, most preferably of the CD4 and/or CD8 phenotypes, but they may be also allogeneic T-cells from related donors, e.g. siblings, parents, children, or HLA-matched or partially matched, semi-allogeneic or fully allogeneic donors.

The NS-specific antiself activated T-cells are preferably non-attenuated, although attenuated NS-specific activated T-cells may be used. T-cells may be attenuated using methods well known in the art, including but not limited to, by gamma-irradiation, e.g. 1.5-10.0 Rads (Ben-Nun, A., Wekerle, H. and Cohen, I. R., Nature 292:60-61 (1981); Ben-Nun, A. and Cohen, I. R., J. Immunol. 129:303-308 (1982)); and/or by pressure treatment, for example as described in U.S. Pat. No. 4,996,194 (Cohen et al.); and/or by chemical cross-linking with an agent such as formaldehyde, glutaraldehyde and the like, for example as described in U.S. Pat. No. 4,996,194 (Cohen et al.); and/or by cross-linking and photoactivation with light with a photoactivatable psoralen compound, for example as described in U.S. Pat. No. 5,114,721 (Cohen et al.); and/or by a cytoskeletal disrupting agent such as cytochalsin and colchicine, for example as described in U.S. Pat. No. 4,996,194 (Cohen et al.). In a preferred embodiment the NS-specific antiself activated T-cells are isolated as described below. T-cells can be isolated and purified according to methods known in the art (Mor and Cohen, 1995, J. Immunol. 155:3693-3699). For an illustrative example, see Section 6.1.

Circulating T-cells of a subject which recognize myelin basic protein or another NS antigen such as the amyloid precursor protein are isolated and expanded using known procedures. In order to obtain NS-specific antiself activated T-cells, T-cells are isolated and the NS-specific ATCs are then expanded by a known procedure (Burns et al., Cell Immunol. 81:435 (1983); Pette et al., Proc. Natl. Acad. Sci. USA 87:7968 (1990); Mortin et al., J. Immunol. 145:540 (1990); Schluesener et al., J. Immunol. 135:3128 (1985); Suruhan-Dires Keneli et al., Euro. J. Immunol. 23:530 (1993) which are incorporated herein by reference in their entirety.

The isolated T-cells may be activated by exposure of the cells to one or more of a variety of natural or synthetic NS-specific antigens or epitopes, including but not limited to, myelin basic protein (MBP), myelin oligodendrocyte glycoprotein (MOG), proteolipid protein (PLP), myelin-associated glycoprotein (MAG), S-100, β-amyloid, Thy-1, P0, P2 and neurotransmitter receptors. In a preferred embodiment, the isolated T cells are activated by one or more cryptic epitopes, including but limited to the following MBP peptides: p11-30, p51-70, p91-110, p131-150, and p151-170.

During ex vivo activation of the T-cells, the T-cells may be activated by culturing them in medium to which at least one suitable growth promoting factor has been added. Growth promoting factors suitable for this purpose include, without limitation, cytokines, for instance tumor necrosis factor α (TNF-α), interleukin 2 (IL-2), and interleukin 4 (IL-4).

In an embodiment, the activated T-cells endogenously produce a substance that ameliorates the effects of injury or disease in the CNS.

In another embodiment, the activated T-cells endogenously produce a substance that stimulates other cells, including, but not limited to, transforming growth factor-β (TGF-β), nerve growth factor (NGF), neurotrophic factor 3 (NT-3), neurotrophic factor 4/5 (NT-4/5), brain-derived neurotrophic factor (BDNF); interferon-γ (IFN-γ), interleukin-6 (IL-6), wherein the other cells, directly or indirectly, ameliorate the effects of injury or disease.

Following their proliferation in vitro, the T-cells are administered to a mammalian subject. In a preferred embodiment, the T-cells are administered to a human subject. T-cell expansion is preferably performed using peptides corresponding to sequences in a non-pathogenic, NS-specific, self protein.

A subject can initially be immunized with a NS-specific antigen using a non-pathogenic peptide of the self protein. A T-cell preparation can be prepared from the blood of such immunized subjects, preferably from T-cells selected for their specificity towards the NS-specific antigen. The selected T-cells can then be stimulated to produce a T-cell line specific to the self-antigen (Ben-Nun et al., J. Immunol. 129:303 (1982)).

The NS-specific antigen may be a purified antigen or a crude NS preparation, as will be described below.

NS-specific antigen activated T-cells, obtained as described above, can be used immediately or may be preserved for later use, e.g. by cryopreservation as described below. NS-specific antiself activated T-cells may also be obtained using previously cryopreserved T-cells, i.e., after thawing the cells, the T-cells may be incubated with NS-specific antigen, optimally together with thymocytes, to obtain a preparation of NS-specific ATCs.

As will be evident to those skilled in the art, the T-cells can be preserved, e.g. by cryopreservation, either before or after culture.

Cryopreservation agents which can be used include but are not limited to dimethyl sulfoxide (DMSO) (Lovelock and Bishop, 1959, Nature 183:1394-1395; Ashwood-Smith, 1961, Nature 190:1204-1205), glycerol, polyvinylpyrrolidone (Rinfret, 1960, Ann. N.Y. Acad. Sci. 85:576), polyethylene glycol (Sloviter and Ravdin, 1962, Nature 196:548), albumin, dextran, sucrose, ethylene glycol, i-erythritol, D-ribitol, D-mannitol (Rowe et al., 1962, Fed. Proc. 21:157), D-sorbitol, i-inositol, D-lactose, choline chloride (Bender et al., 1960, J. Appl. Physiol. 15:520), amino acids (Phan The Tran and Bender, 1960, Exp. Cell Res. 20:651), methanol, acetamide, glycerol monoacetate (Lovelock, 1954, Biochem. J. 56:265), inorganic salts (Phan The Tran and Bender, 1960, Proc. Soc. Exp. Biol. Med. 104:388; Phan The Tran and Bender, 1961, in Radiobiology, Proceedings of the Third Australian Conference on Radiobiology, Ilbery, P.L.T., ed., Butterworth, London, p. 59), and DMSO combined with hydroxyethel starch and human serum albumin (Zaroulis and Leiderman, 1980, Cryobiology 17:311-317).

A controlled cooling rate is critical. Different cryoprotective agents (Rapatz et al., 1968, Cryobiology 5(1):18-25) and different cell types have different optimal cooling rates. See, e.g., Rowe and Rinfret, 1962, Blood 20:636; Rowe, 1966, Cryobiology 3(1):12-18; Lewis et al., 1967, Transfusion 7(1):17-32; and Mazur, 1970, Science 168:939-949 for effects of cooling velocity on survival of cells and on their transplantation potential. The heat of fusion phase where water turns to ice should be minimal. The cooling procedure can be carried out by use of, e.g., a programmable freezing device or a methanol bath procedure.

Programmable freezing apparatuses allow determination of optimal cooling rates and facilitate standard reproducible cooling. Programmable controlled-rate freezers such as Cryomed or Planar permit tuning of the freezing regimen to the desired cooling rate curve.

After thorough freezing, cells can be rapidly transferred to a long-term cryogenic storage vessel. In one embodiment, samples can be cryogenically stored in mechanical freezers, such as freezers that maintain a temperature of about −80° C. or about −20° C. In a preferred embodiment, samples can be cryogenically stored in liquid nitrogen (−196° C.) or its vapor. Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum.

Considerations and procedures for the manipulation, cryopreservation, and long term storage of T-cells can be found, for example, in the following references, incorporated by reference herein: Gorin, 1986, Clinics in Haematology 15(1):19-48; Bone-Marrow Conservation, Culture and Transplantation, Proceedings of a Panel, Moscow, Jul. 22-26, 1968, International Atomic Energy Agency, Vienna, pp. 107-186.

Other methods of cryopreservation of viable cells, or modifications thereof, are available and envisioned for use, e.g., cold metal-mirror techniques. See Livesey and Linner, 1987, Nature 327:255; Linner et al., 1986, J. Histochem. Cytochem. 34(9):1123-1135; see also U.S. Pat. No. 4,199,022 by Senken et al., U.S. Pat. No. 3,753,357 by Schwartz, U.S. Pat. No. 4,559,298 by Fahy.

Frozen cells are preferably thawed quickly (e.g., in a water bath maintained at 37-41° C.) and chilled immediately upon thawing. It may be desirable to treat the cells in order to prevent cellular clumping upon thawing. To prevent clumping, various procedures can be used, including but not limited to the addition before or after freezing of DNAse (Spitzer et al., 1980, Cancer 45:3075-3085), low molecular weight dextran and citrate, hydroxyethyl starch (Stiff et al., 1983, Cryobiology 20:17-24), or acid citrate dextrose (Zaroulis and Leiderman, 1980, Cryobiology 17:311-317), etc.

The cryoprotective agent, if toxic in humans, should be removed prior to therapeutic use of the thawed T-cells. One way in which to remove the cryoprotective agent is by dilution to an insignificant concentration.

Once frozen T-cells have been thawed and recovered, they are used to promote axonal regeneration as described herein with respect to non-frozen T-cells.

### 5.2 NS-Specific Antigens and Peptides Derived Therefrom

Pharmaceutical compositions comprising a NS-specific antigen or peptide derived therefrom or derivative thereof can be used for preventing or inhibiting the effects of injury or disease that result in NS degeneration or for promoting nerve regeneration in the NS, particularly in the CNS. Additionally, NS-specific antigens or peptides derived therefrom or derivatives thereof may be used for in vivo or in vitro activation of antiself T-cells. In an embodiment, the NS-specific antigen is an isolated or purified antigen. In an embodiment, methods of promoting nerve regeneration or of preventing or inhibiting the effects of CNS or PNS injury or disease comprise administering NS-specific antigen or a peptide derived therefrom or derivative thereof to a mammal wherein the NS-specific antigen or peptide derived therefrom or derivative thereof activates T-cells in vivo to produce a population of T-cells that accumulate at a site of injury or disease of the CNS or PNS.

The NS-specific antigen may be an antigen obtained from NS tissue, preferably from tissue at a site of CNS injury or disease. The NS-specific antigen may be isolated and purified by standard methods including chromatography (e.g., ion exchange, affinity, and sizing column chromatography), centrifugation, differential solubility, or by any other standard technique for the purification of antigens. The functional properties may be evaluated using any suitable assay. In the practice of the invention, natural or synthetic NS-specific antigens or epitopes include, but are not limited to, MBP, MOG, PLP, MAG, S-100, β-amyloid, Thy-1, P0, P2 and a neurotransmitter receptor.

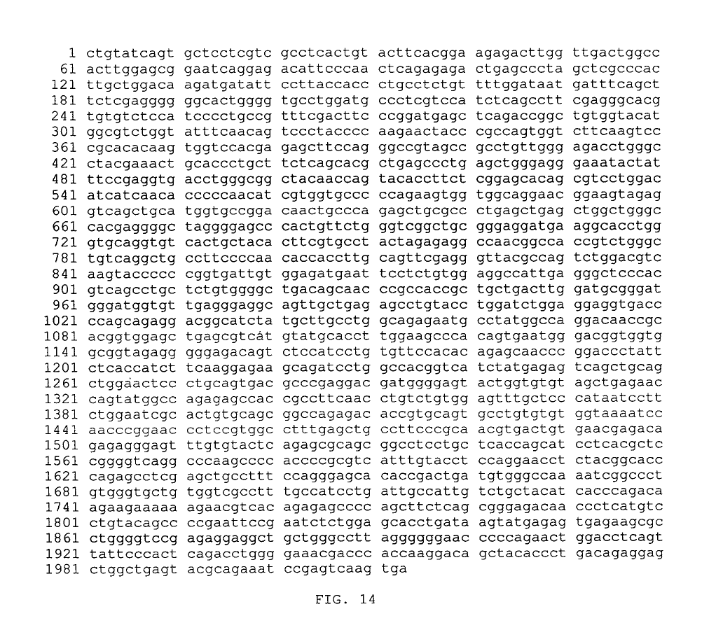

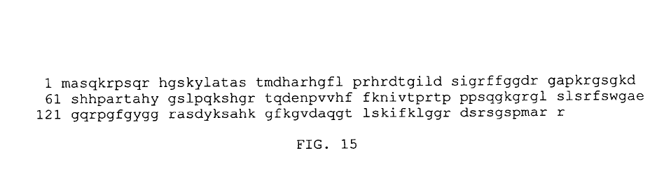

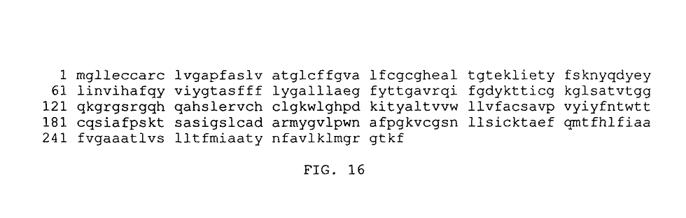

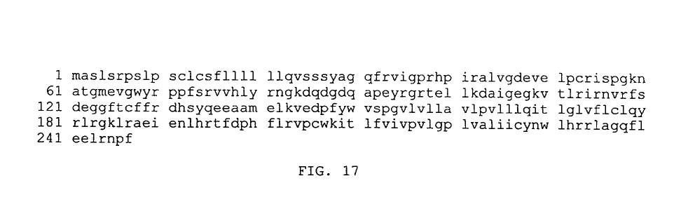

Specific illustrative examples of useful NS-specific antigens include but are not limited to, human MBP, depicted in FIG. 15 (SEQ ID NO: ______); human proteolipid protein, depicted in FIG. 16 (SEQ ID NO: ______); and human oligodendrocyte glycoprotein, depicted in FIG. 17 (SEQ ID NO: ______).

In a preferred embodiment, peptides derived from NS-specific, self antigens or derivatives of NS-specific antigens activate T-cells, but do not induce an autoimmune disease. An example of such peptide is a peptide comprising amino acids 51-70 of myelin basic protein. SEQ ID NO: ______(Kamholz et al., 1986, Proc. Natl. Acad. Sci. U.S.A. 83:4962-4966, GenBank accession number M13577; Roth et al., 1987, J. Neurosci. Res. 17(4):321-328, GenBank accession number M30516).

In addition, a NS-specific antigen may be a crude NS-tissue preparation, e.g., derived from NS tissue obtained from mammalian NS. Such a preparation may include cells, both living or dead cells, membrane fractions of such cells or tissue, etc.

A NS-specific antigen may be obtained by a NS biopsy or necropsy from a mammal including, but not limited to, from a site of CNS injury; from cadavers; from cell lines grown in culture. Additionally, a NS-specific antigen may be a protein obtained by genetic engineering, chemically synthesized, etc.

In addition to NS-specific antigens, the invention also relates to peptides derived from NS-specific antigens or derivatives including chemical derivatives and analogs of NS-specific antigens which are functionally active, i.e., they are capable of displaying one or more known functional activities associated with a full-length NS-specific antigen. Such functional activities include but are not limited to antigenicity [ability to bind (or compete with a CNS-antigen for binding) to an anti-NS-specific antibody], immunogenicity (ability to generate antibody which binds to a NS-specific protein), and ability to interact with T-cells, resulting in activation comparable to that obtained using the corresponding full-length antigen.

A peptide derived from a CNS-specific or PNS-specific antigen has a sequence comprised within the antigen sequence and is either: (1) an immunogenic peptide, i.e., a peptide that can elicit a human T-cell response detected by T-cell proliferation or by cytokine (e.g. interferon (IFN)-γ, interleukin (IL) -2, IL-4 or IL-10) production or (2) a “cryptic epitope” (also designated herein as “immunosilent” or “nonimmunodominant” epitope), i.e., a peptide that by itself can induce a T-cell immune response that is not induced by the whole antigen protein (see Moalem et al., 1999, Nature Med. 5(1)). Cryptic epitopes for use in the present invention include, but are not limited to, peptides of the myelin basic protein sequence: peptide p11-30, p51-70, p91-110, p131-150, and p151-170. Other peptides can be identified by their capacity to elicit a human T-cell response detected by T-cell proliferation or by cytokine (e.g. IFN-γ, IL-2, IL-4, or IL-10) production.

In a specific embodiment of the invention, peptides consisting of or comprising a fragment of a NS-specific antigen consisting of at least 10 (contiguous) amino acids of the NS-specific antigen is provided. In other embodiments, the fragment consists of at least 20 contiguous amino acids or 50 contiguous amino acids of the NS-specific antigen.

Derivatives of a NS-specific antigen also include but are not limited to those molecules comprising regions that are substantially homologous to the full-length antigen or fragments thereof (e.g., in various embodiments, at least 60% or 70% or 80% or 90% or 95% identity over an amino acid sequence of identical size or when compared to an aligned sequence in which the alignment is done by a computer homology program known in the art) or whose encoding nucleic acid is capable of hybridizing to a coding nucleotide sequence of the full-length NS-specific antigen, under high stringency, moderate stringency, or low stringency conditions.

Computer programs for determining homology may include but are not limited to TBLASTN, BLASTP, FASTA, TFASTA, and CLUSTALW (Pearson and Lipman, 1988, Proc. Natl. Acad. Sci. USA 85(8):2444-8; Altschul et al., 1990, J. Mol. Biol. 215(3):403-10; Thompson, et al., 1994, Nucleic Acids Res. 22(22):4673-80; Higgins, et al., 1996, Methods Enzymol 266:383-402; Altschul, et al., 1990, J. Mol. Biol. 215(3):403-10).

The NS-specific antigen derivatives of the invention can be produced by various methods known in the art. The manipulations which result in their production can occur at the gene or protein level. For example, a cloned gene sequence can be modified by any of numerous strategies known in the art (Maniatis, T., 1990, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro.

Additionally, the coding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, chemical mutagenesis, in vitro site-directed mutagenesis (Hutchinson, C., et al., 1978, J. Biol. Chem 253:6551), etc.

Manipulations may also be made at the protein level. Included within the scope of the invention are derivatives which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

In addition, derivatives of a NS-specific antigen can be chemically synthesized. For example, a peptide corresponding to a portion of an antigen which comprises the desired domain or which mediates the desired activity can be synthesized by use of a peptide synthesizer. Furthermore, if desired, nonclassical amino acids or chemical amino acid analogs can be introduced as a substitution or addition into the amino acid sequence. Non-classical amino acids include but are not limited to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

The functional activity of NS-specific antigens and peptides derived therefrom and derivatives thereof can be assayed by various methods known in the art, including, but not limited to T-cell proliferation assays (Mor and Cohen, 1995, J. Immunol. 155:3693-3699).

A NS-specific antigen or peptide derived therefrom or derivative thereof may be kept in solution or may be provided in a dry form, e.g. as a powder or lyophilizate, to be mixed with appropriate solution prior to use.

### 5.3 Nucleotide Sequences Encoding NS-Antigens and Peptides Derived Therefrom

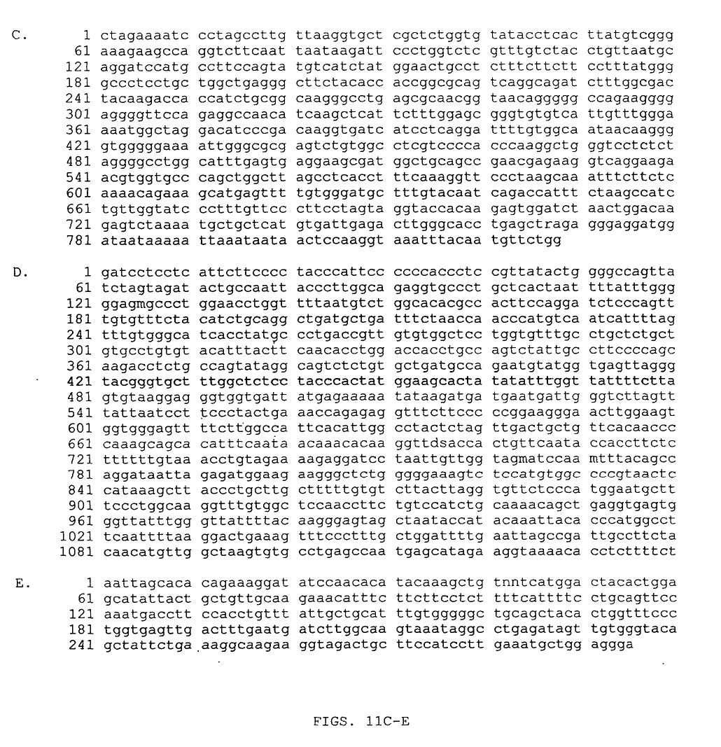

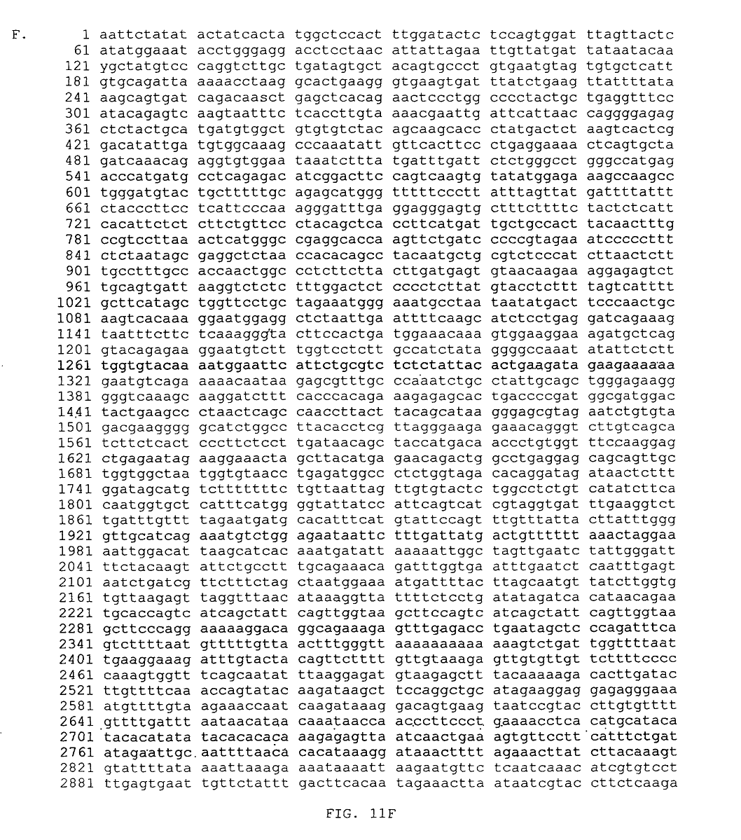

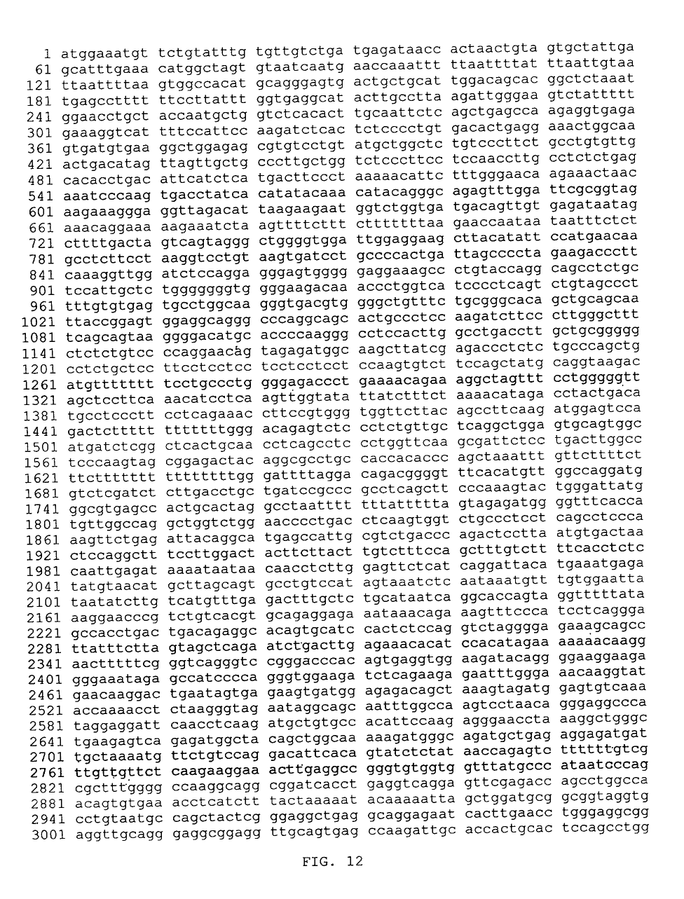

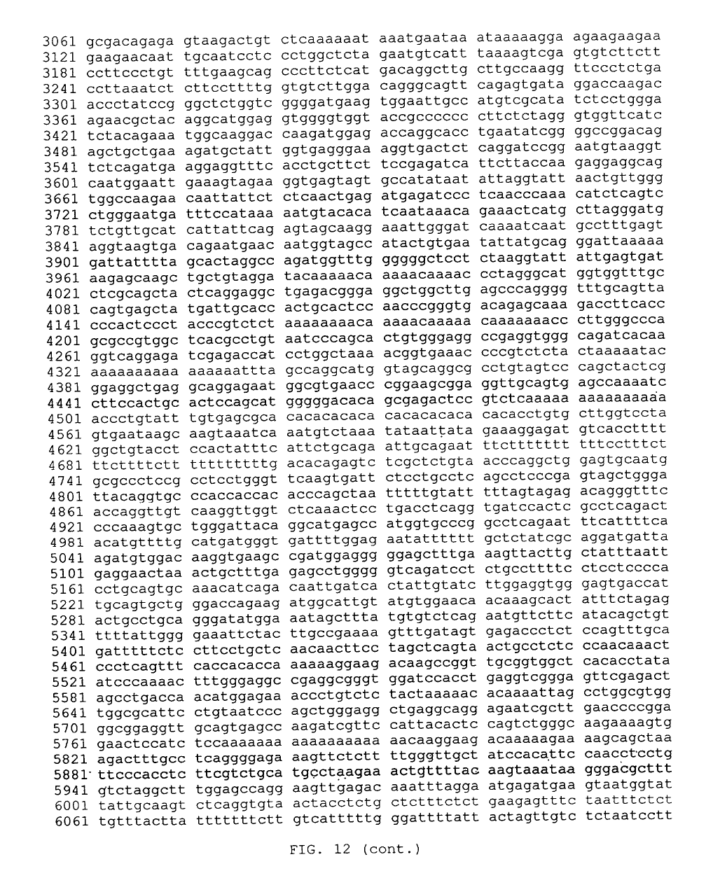

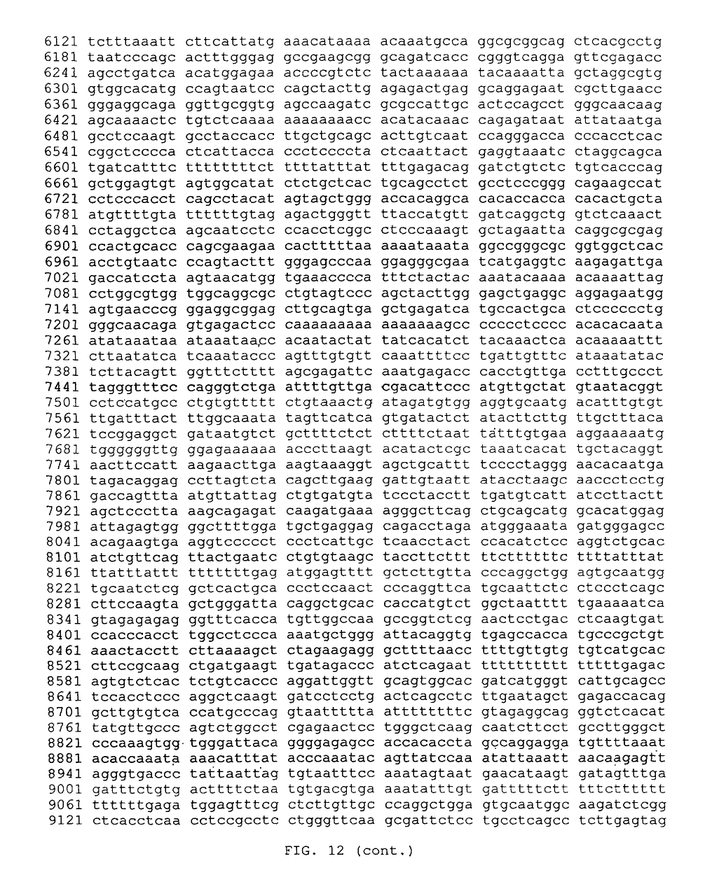

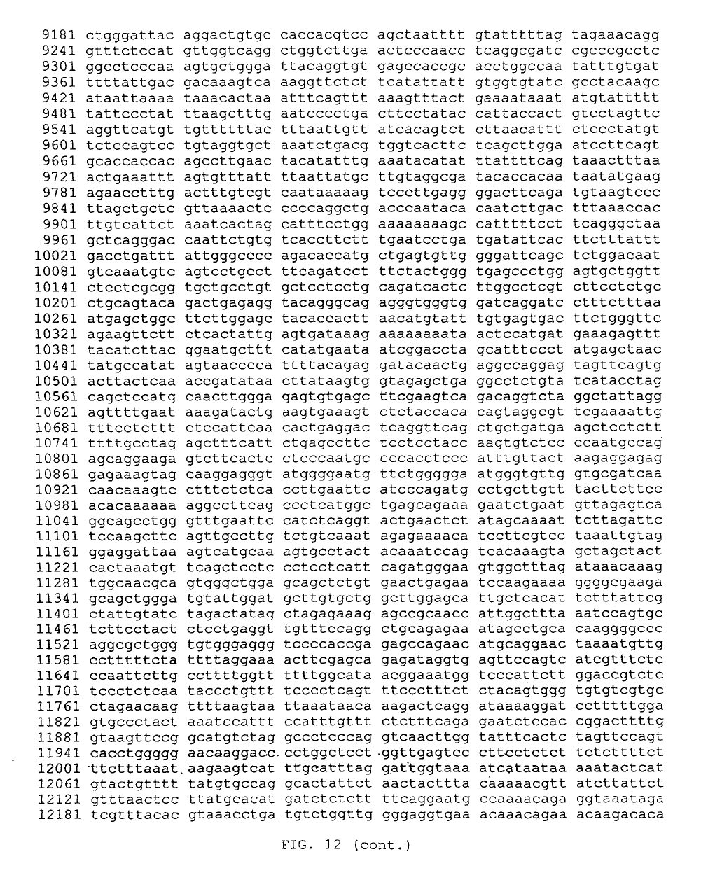

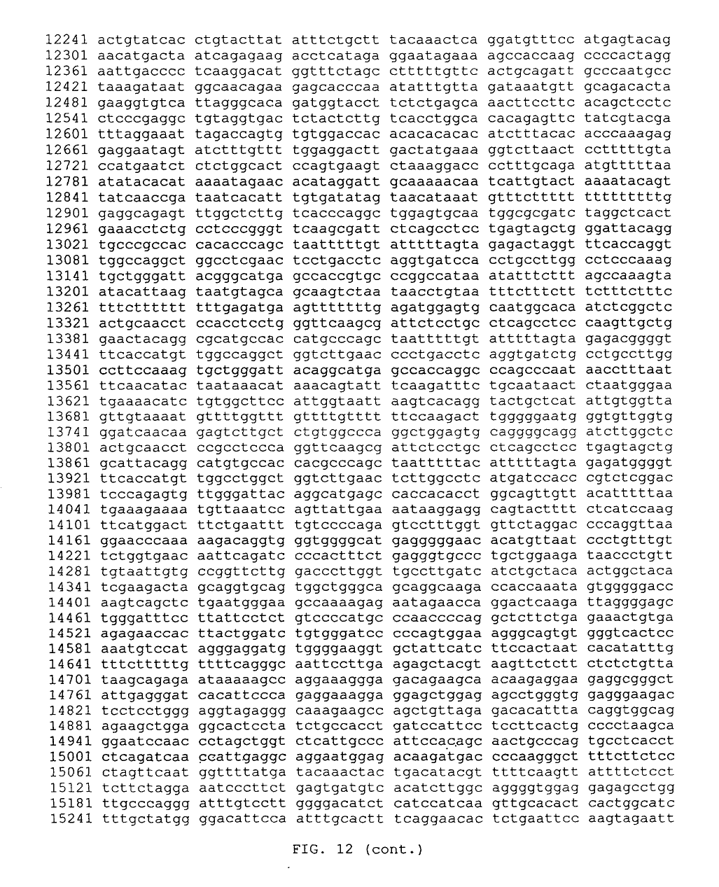

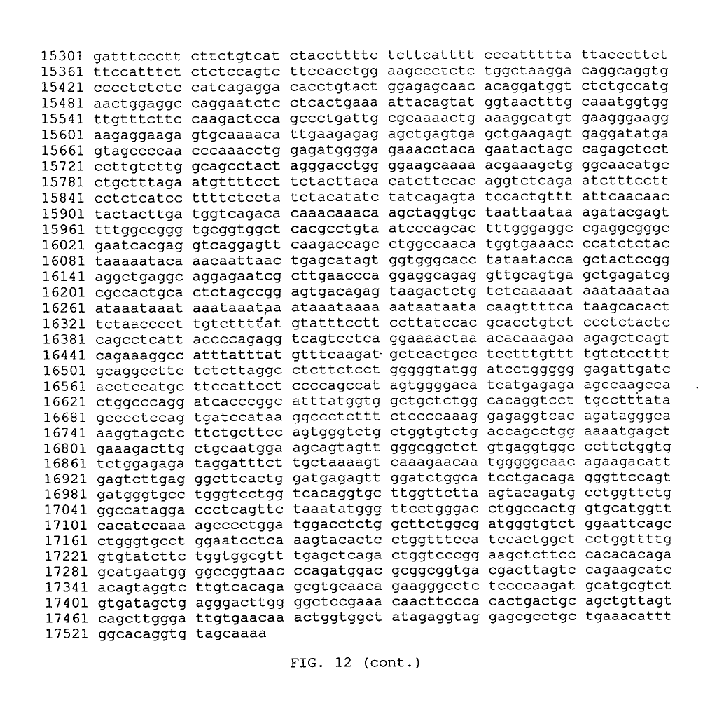

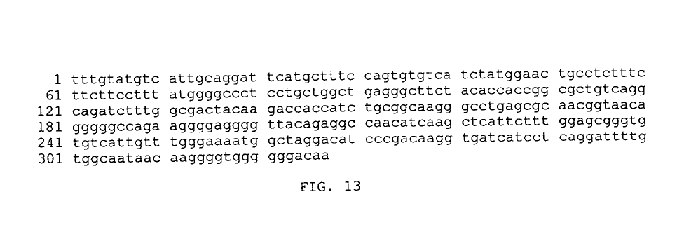

Compositions comprising a nucleotide sequence encoding a NS-specific antigen or peptide derived therefrom can be used for preventing or inhibiting the effects of injury or disease that result in CNS or PNS degeneration or for promoting nerve regeneration in the CNS or PNS. Specific illustrative examples of useful nucleotide sequences encoding NS-specific antigens or peptides derived from a NS-specific antigen, include but are not limited to nucleotide sequences encoding rat myelin basic protein (MBP) peptides, depicted in FIG. 9 (SEQ ID NO: ______); human MBP, depicted in FIG. 10 (SEQ ID NO: ______); human myelin PLP, depicted in FIGS. 11(A-F) (SEQ ID NO: ______); human MOG, depicted in FIG. 12 (SEQ ID NO: ______); rat PLP and variant, depicted in FIG. 13 (SEQ ID NO: ______); and rat MAG, depicted in FIG. 14 (SEQ ID NO: ______).

### 5.4 Therapeutic Uses

The compositions described in Sections 5.1 through 5.3 may be used to promote nerve regeneration or to prevent or inhibit secondary degeneration which may otherwise follow primary NS injury, e.g. blunt trauma, penetrating trauma, hemorrhagic stroke, ischemic stroke or damages caused by surgery such as tumor excision. In addition, such compositions may be used to ameliorate the effects of disease that result in a degenerative process, e.g. degeneration occurring in either grey or white matter (or both) as a result of various diseases or disorders which are not recognized by those of reasonable skill in the art as being autoimmune diseases or disorders including, without limitation: Diabetic neuropathy, senile dementias, Alzheimer's disease, Parkinson's Disease, facial nerve (Bell's) palsy, glaucoma, Huntington's chorea, amyotrophic lateral sclerosis (ALS), non-arteritic optic neuropathy, intervertebral disc herniation, vitamin deficiency, prion diseases such as Creutzfeldt-Jakob disease, carpal tunnel syndrome, peripheral neuropathies associated with various diseases, including but not limited to, uremia, porphyria, hypoglycemia, Sjörgren-Larsson syndrome, acute sensory neuropathy, chronic ataxic neuropathy, biliary cirrhosis, primary amyloidosis, obstructive lung diseases, acromegaly, malabsorption syndromes, polycythemia vera, IgA and IgG gammapathies, complications of various drugs (e.g. metronidazole) and toxins (e.g. alcohol or organophosphates), Charcot-Marie-Tooth disease, ataxia telangectasia, Friedreich's ataxia, amyloid polyneuropathies, adrenomyeloneuropathy, Giant axonal neuropathy, Refsum's disease, Fabry's disease, lipoproteinemia, etc.

In a preferred embodiment, the NS-specific antiself activated T-cells, the NS-specific antigens, peptides derived therefrom, derivatives thereof or the nucleotides encoding said antigens, or peptides or any combination thereof of the present invention are used to treat diseases or disorders which are not autoimmune diseases or neoplasias. In a preferred embodiment, the compositions of the present invention are administered to a human subject.

### 5.5 Formulations and Administration

Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

The term “carrier” refers to a diluent, adjuvant, excipient, or vehicle with which the therapeutic is administered. The carriers in the pharmaceutical composition may comprise a binder, such as microcrystalline cellulose, polyvinylpyrrolidone (polyvidone or povidone), gum tragacanth, gelatine, starch, lactose or lactose monohydrate; a disintegrating agent, such as alginic acid, maize starch and the like; a lubricant or surfactant, such as magnesium stearate, or sodium lauryl sulphate; a glidant, such as colloidal silicon dioxide; a sweetening agent, such as sucrose or saccharin; and/or a flavoring agent, such as peppermint, methyl salicylate, or orange flavoring.

Methods of administration include, but are not limited to, parenteral, e.g. intravenous, intraperitoneal, intramuscular, subcutaneous, mucosal (e.g., oral, intranasal, buccal, vaginal, rectal, intraocular), intrathecal and intradermal routes. Administration can be systemic or local.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the active compound.

For buccal administration, the compositions may take the form of tablets or lozenges formulated in conventional manner.

The compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions may also be formulated in rectal compositions such as suppositories or retention enemas, e.g., containing conventional suppository bases such as cocoa butter or other glycerides.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

In a preferred embodiment, compositions comprising NS-specific antiself activated T-cells, a NS-specific antigen or peptide derived therefrom, or derivative thereof, or a nucleotide sequence encoding such antigen or peptide are formulated in accordance with routine procedures as pharmaceutical compositions adapted for intravenous or intraperitoneal administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

Pharmaceutical compositions comprising NS-specific antigen or peptide derived therefrom or derivative thereof may optionally be administered with an adjuvant, such as Incomplete Freund's Adjuvant.

The invention also provides a pharmaceutical pack or kit comprising one or more containers filled with one or more of the ingredients of the pharmaceutical compositions of the invention.

In a preferred embodiment, the pharmaceutical compositions of the invention are administered to a mammal, preferably a human, shortly after injury or detection of a degenerative lesion in the NS. The therapeutic methods of the invention may comprise administration of a NS-specific antiself activated T-cell, or a NS-specific antigen or peptide derived therefrom or derivative thereof, or a nucleotide sequence encoding such antigen or peptide or any combination thereof. The NS-specific antigen may be administered before, concurrently or after administration of NS-specific antiself activated T-cells, a peptide derived from a NS-specific antigen or derivative thereof or a nucleotide sequence encoding such antigen or peptide.

In an embodiment, the compositions of the invention are administered in combination with one or more of the following: (a) mononuclear phagocytes, preferably cultured monocytes (as described in PCT publication No. WO 97/09985, which is incorporated herein by reference in its entirety), that have been stimulated to enhance their capacity to promote axonal regeneration; (b) a neurotrophic factor such as acidic fibroblast growth factor; and (c) an anti-inflammatory therapeutic substance (i.e., an anti-inflammatory steroid, such as dexamethasone or methylprednisolone, or a non-steroidal anti-inflammatory agent or drug, such as aspirin, indomethacin, ibuprofen, fenoprofen, ketoprofen or haproxen, or an anti-inflammatory peptide, such as Thr-Lys-Pro (TKP)).

In an embodiment, mononuclear phagocyte cells according PCT Publication No. WO 97/09985 and U.S. patent application Ser. No. 09/041,280, filed Mar. 11, 1998, are injected into the site of injury or lesion within the CNS, either concurrently, prior to, or following parenteral administration of NS-specific antiself activated T-cells, a NS-specific antigen or peptide derived therefrom or derivative thereof, or a nucleotide sequence encoding such antigen or peptide.

In an embodiment, administration of NS-specific activated T-cells, a NS-specific antigen or peptide derived therefrom or derivative thereof, or a nucleotide sequence encoding such antigen or peptide, may be administered as a single dose or may be repeated, preferably at 2 week intervals and then successively longer intervals once a month, once a quarter, once every six months, etc. The course of treatment may last several months, several years or occasionally also through the life-time of the individual, depending on the condition or disease which is being treated. In the case of a CNS injury, the treatment may range between several days to months or even years, until the condition has stabilized and there is no or only a limited risk of development of secondary degeneration. In chronic human diseases or conditions such as Alzheimer's disease or Parkinson's disease, the therapeutic treatment in accordance with the invention may be for life.

As will be evident to those of skill in the art, the therapeutic effect depends at times on the condition or disease to be treated, on the individual's age and health condition, on other physical parameters (e.g. gender, weight, etc.) of the individual, as well as on various other factors, e.g. whether the individual is taking other drugs, etc.

The optimal dose of the therapeutic compositions comprising NS-specific antiself activated T-cells of the invention is proportional to the number of nerve fibers affected by CNS injury or disease at the site being treated. In a preferred embodiment, the dose ranges from about 5×10⁶ to about 10⁷ for treating a lesion affecting about 10⁵ nerve fibers, such as a complete transection of a rat optic nerve, and ranges from about 10⁷ to about 10⁸ for treating a lesion affecting about 10⁶-10⁷ nerve fibers, such as a complete transection of a human optic nerve. As will be evident to those of skill in the art, the dose of T-cells can be scaled up or down in proportion to the number of nerve fibers thought to be affected at the lesion or site of injury being treated.

The following examples illustrate certain features of the present invention but are not intended to limit the scope of the present invention.

### 6. EXAMPLE

### Accumulation of Activated T-Cells in Injured Optic Nerve

### 6.1 Materials and Methods

### 6.1.1 Animals

Female Lewis rats were supplied by the Animal Breeding Center of the Weizmann Institute of Science (Rehovot, Ill.), matched for age (8-12 weeks) and housed four to a cage in a light and temperature-controlled room.

### 6.1.2 Media

The T-cell proliferation medium contained the following: Dulbecco's modified Eagle's medium (DMEM, Biological Industries, Israel) supplemented with 2 mM L-glutamine (L-Glu, Sigma, USA), 5×10⁻⁵M 2-mercaptoethanol (2-ME, Sigma), penicillin (100 IU/ml; Biological Industries), streptomycin (100 μg/ml; Biological Industries), sodium pyruvate (1 mM; Biological Industries), non-essential amino acids (1 ml/100 ml; Biological Industries) and autologous rat serum 1% (vol/vol) (Mor et al., Clin. Invest., 85:1594 (1990)). Propagation medium contained: DMEM, 2-ME, L-Glu, sodium pyruvate, non-essential amino acids and antibiotics in the same concentration as above with the addition of 10% fetal calf serum (FCS), and 10% T-cell growth factor (TCGF) obtained from the supernatant of concanavalin A-stimulated spleen cells (Mor et al., supra, 1990).

### 6.1.3 Antigens

Myelin basic protein (MBP) from the spinal cords of guinea pigs was prepared as described (Hirshfeld, et al., 1970, FEBS Lett. 7:317). Ovalbumin was purchased from Sigma (St. Louis, Mo.). The p51-70 of the rat 18.5 kDa isoform of MBP (sequence: APKRGSGKDSHTRTTHYG) SEQ ID NO:______ and the p277 peptide of the human hsp60 (sequence: VLGGGCALLRCPALDSLTPANED) SEQ ID NO:______ (Elias, et al., 1991, Proc. Natl. Acad. Sci. USA 88, 3088-91) were synthesized using the 9-fluorenylmethoxycarbonyl technique with an automatic multiple peptide synthesizer (AMS 422, ABIMED, Langenfeld, Germany). The purity of the peptides was analyzed by HPLC and amino acid composition.

### 6.1.4 T-Cell Lines

T-cell lines were generated from draining lymph node cells obtained from Lewis rats immunized with an antigen (described above in Section 6.1.3). The antigen was dissolved in PBS (1 mg/ml) and emulsified with an equal volume of incomplete Freund's adjuvant (Difco Laboratories, Detroit, Mich.) supplemented with 4 mg/ml Mycobacterium tuberculosis (Difco Laboratories, Detroit, Mich.). The emulsion (0.1 ml) was injected into hind foot pads of the rats. Ten days after the antigen was injected, the rats were killed and draining lymph nodes were surgically removed and dissociated. The cells were washed and activated with the antigen (10 μg/ml) in proliferation medium (described above in Section 6.1.2). After incubation for 72 h at 37° C., 90% relative humidity and 7% CO₂, the cells were transferred to propagation medium (described above in Section 6.1.2). Cells were grown in propagation medium for 4-10 days before being re-exposed to antigen (10 μg/ml) in the presence of irradiated (2000 rad) thymus cells (10⁷ cells/ml) in proliferation medium. The T-cell lines were expanded by repeated re-exposure and propagation.

### 6.1.5 Crush Injury of Rat Optic Nerve

Crush injury of the optic nerve was performed as previously described (Duvdevani et al., 1990, Neurol. Neurosci. 2:31-38). Briefly, rats were deeply anesthetized by i.p. injection of Rompun (xylazine, 10 mg/kg; Vitamed, Israel) and Vetalar (ketamine,50 mg/kg; Fort Dodge Laboratories, Fort Dodge, Iowa). Using a binocular operating microscope, a lateral canthotomy was performed in the right eye and the conjunctiva was incised lateral to the cornea. After separation of the retractor bulbi muscles, the optic nerve was exposed intraorbitally by blunt dissection. Using calibrated cross-action forceps, a moderate crush injury was inflicted on the optic nerve, 2 mm from the eye (Duvdevani et al., Instructure Neurology and Neuroscience, 2:31, 1990). The contralateral nerve was left undisturbed and was used as a control.

### 6.1.6 Immunocytochemistry of T-Cells

Longitudinal cryostat nerve sections (20 μm thick) were picked up onto gelatin glass slides and frozen until preparation for fluorescent staining. Sections were thawed and fixed in ethanol for 10 minutes at room temperature, washed twice with double-distilled water (ddH₂O), and incubated for 3 minutes in PBS containing 0.05% polyoxyethylene-sorbitan monolaurate (Tween-20; Sigma, USA). Sections were then incubated for 1 hr at room temperature with a mouse monoclonal antibody directed against rat T-cell receptor (TCR) (1:100, Hunig et al., J. Exp. Med., 169:73, 1989), in PBS containing 3% FCS and 2% BSA. After three washes with PBS containing 0.05% Tween-20, the sections were incubated with fluorescein isothiocyanate-conjugated goat anti-mouse IgG (with minimal cross-reaction to rat, human, bovine and horse serum proteins) (Jackson ImmunoResearch, West Grove, Pa.) for 1 hr at room temperature. The sections were then washed with PBS containing Tween-20 and treated with glycerol containing 1,4-diazobicyclo-(2,2,2) octane (Sigma), to inhibit quenching of fluorescence. The sections were viewed with a Zeiss microscope and cells were counted. Staining in the absence of first antibody was negative.

### 6.2 Results

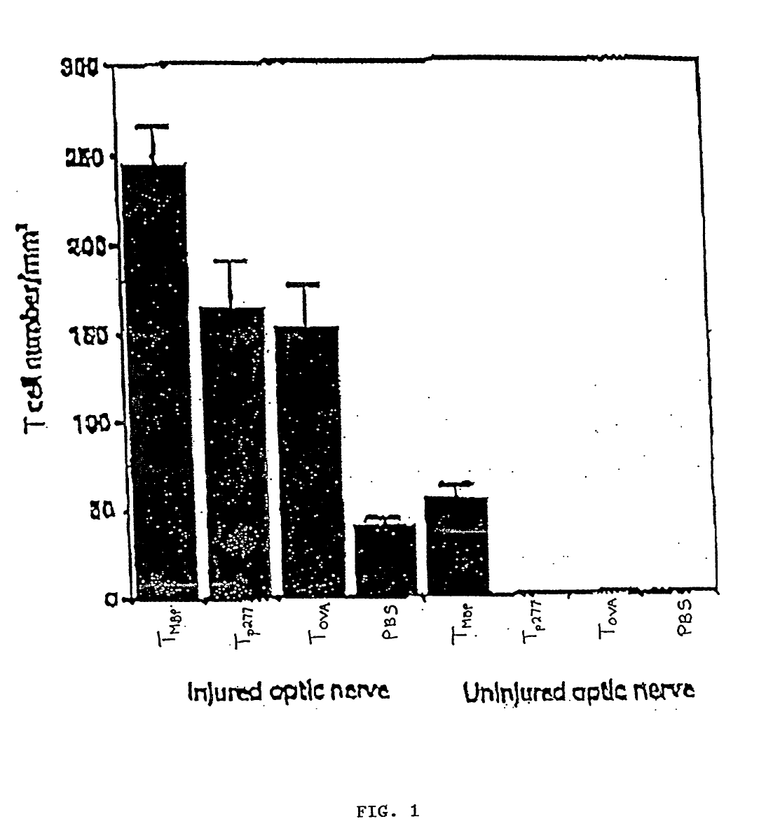

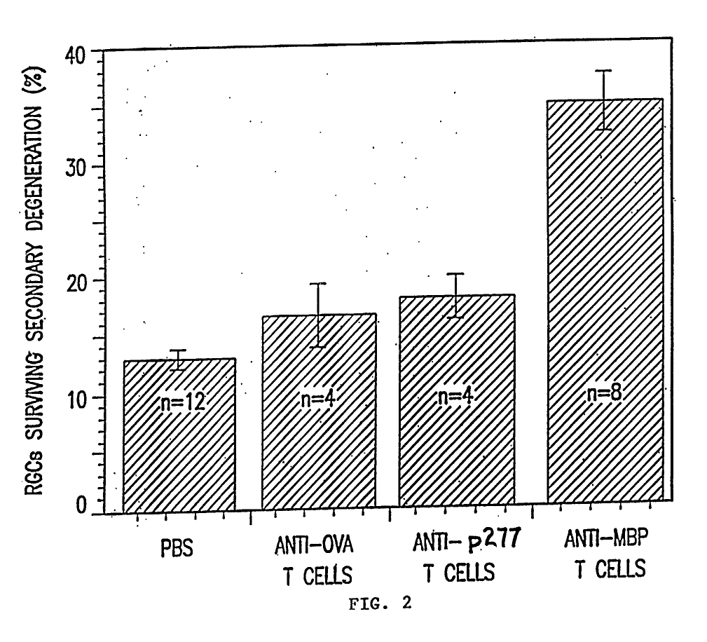

FIG. 1 shows accumulation of T-cells measured immuno-histochemically. The number of T cells was considerably higher in injured nerves of rats injected with anti-MBP, anti-OVA or anti-p277 cells; statistical analysis (one-way ANOVA) showed significant differences between T cell numbers in injured optic nerves of rats injected with anti-MBP, anti-OVA, or anti-p277 T cells and in injured optic nerves of rats injected with PBS (P<0.001); and between injured optic nerves and uninjured optic nerves of rats injected with anti-MBP, anti-OVA, or anti-p277 T cells (P<0.001).

### 7. EXAMPLE

### Neuroprotection by Autoimmune Anti-MBP T-cells

### 7.1 Materials and Methods

Animals, media, antigens, crush injury of rat optic nerve, sectioning of nerves, T-cell lines, and immunolabeling of nerve sections are described in Section 6, supra.

### 7.1.1 Retrograde Labeling and Measurement of Primary Damage and Secondary Degeneration

Primary damage of the optic nerve axons and their attached retinal ganglion cells (RGCs) were measured after the immediate post-injury application of the fluorescent lipophilic dye 4- (4-(didecylamino)styryl)-n-methylpyridinium iodide (4-Di-10-Asp) (Molecular Probes Europe BV, Netherland) distal to the site of injury. Only axons that are intact are capable of transporting the dye back to their cell bodies; therefore, the number of labeled cell bodies is a measure of the number of axons that survived the primary damage. Secondary degeneration was also measured by application of the dye distal to the injury site, but 2 weeks after the primary lesion was inflicted. Application of the neurotracer dye distal to the site of the primary crush after 2 weeks ensures that only axons that survived both the primary damage and the secondary degeneration will be counted. This approach makes it possible to differentiate between neurons that are still functionally intact and neurons in which the axons are injured but the cell bodies are still viable, as only those neurons whose fibers are morphologically intact can take up dye applied distally to the site of injury and transport it to their cell bodies. Using this method, the number of labeled ganglion cells reliably reflects the number of still-functioning neurons. Labeling and measurement were done by exposing the right optic nerve for a second time, again without damaging the retinal blood supply. Complete axotomy was done 1-2 mm from the distal border of the injury site and solid crystals (0.2-0.4 mm in diameter) of 4-Di-10-Asp were deposited at the site of the newly formed axotomy. Uninjured optic nerves were similarly labeled at approximately the same distance from the globe. Five days after dye application, the rats were killed. The retina was detached from the eye, prepared as a flattened whole mount in 4% paraformaldehyde solution and examined for labeled ganglion cells by fluorescence microscopy. The percentage of RGCs surviving secondary degeneration was calculated using the following formula: (Number of spared neurons after secondary degeneration)/(Number of spared neurons after primary damage)×100.

### 7.1.2 Electrophysiological Recordings

Nerves were excised and their compound action potentials (CAPs) were recorded in vitro using a suction electrode experimental set-up (Yoles, E. et al., 1996, J. Neurotrauma, 13:49-57). At different times after injury and injection of T cells or PBS, rats were killed by intraperitoneal injection of pentobarbitone (170 mg/kg) (CTS Chemical Industries, Israel). Both optic nerves were removed while still attached to the optic chiasma, and were immediately transferred to a vial containing a fresh salt solution consisting of 126 mM NaCl, 3 mM KCl, 1.25 mM NaH₂PO₂ 26 mM NaHCO₂ 2 mM MgSO₄, 2 mM CaCl₂ and 10 mM D-glucose, aerated with 95% O₂ and 5% CO₂ at room temperature. After 1 hour, electrophysiological recordings were made. In the injured nerve, recordings were made in a segment distal to the injury site. This segment contains axons of viable retinal ganglion cells that have escaped both primary and secondary damage, as well as the distal stumps of non-viable retinal ganglion cells that have not yet undergone Wallerian degeneration. The nerve ends were connected to two suction Ag-AgCl electrodes immersed in the bathing solution at 37° C. A stimulating pulse was applied through the electrode, and the CAP was recorded by the distal electrode. A stimulator (SD9; Grass Medical Instruments, Quincy, Mass.) was used for supramaximal electrical stimulation at a rate of 1 pps to ensure stimulation of all propagating axons in the nerve. The measured signal was transmitted to a microelectrode AC amplifier (model 1800; A-M Systems, Everett, Wash.). The data were processed using the LabView 2.1.1 data acquisition and management system (National Instruments, Austin, Tex.). For each nerve, the difference between the peak amplitude and the mean plateau of eight CAPs was computed and was considered as proportional to the number of propagating axons in the optic nerve. The experiments were done by experimenters ‘blinded’ to sample identity. In each experiment the data were normalized relative to the mean CAP of the uninjured nerves from PBS-injected rats.

### 7.1.3 Clinical Evaluation of Experimental Autoimmune Encephalomyelitis

Clinical disease was scored every 1 to 2 days according to the following neurological scale: 0, no abnormality; 1, tail atony; 2, hind limb paralysis; 3, paralysis extending to thoracic spine; 4, front limb paralysis; 5, moribund state.

### 7.2 Results

### 7.2.1 Neuroprotection by Autoimmune Anti-MBP T-Cells

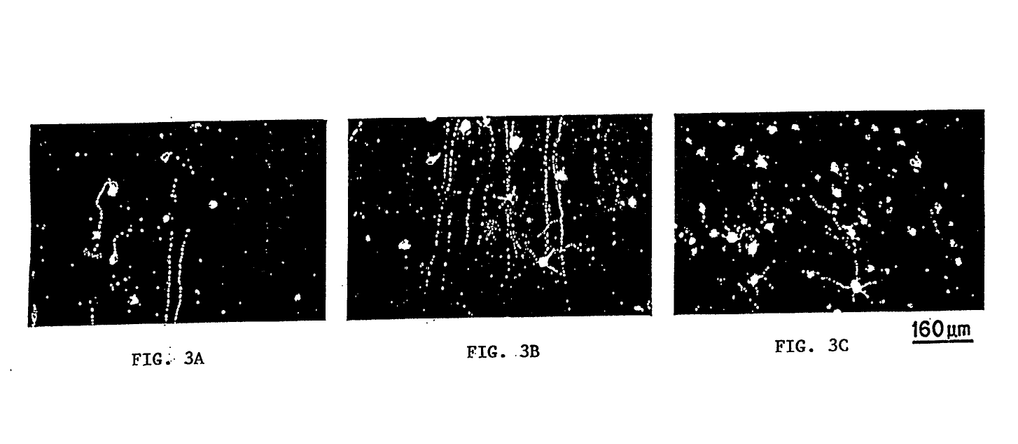

Morphological analyses were done to assess the effect of the T cells on the response of the nerve to injury, and specifically on secondary degeneration. Rats were injected itraperitoneally immediately after optic nerve injury with PBS or with 1×10⁷ activated T cells of the various cell lines. The degree of primary damage to the optic nerve axons and their attached RGCs was measured by injecting the dye 4-Di-10-Asp distal to the site of the lesion immediately after the injury. A time lapse of 2 weeks between a moderate crush injury and dye application is optimal for demonstrating the number of still-viable labeled neurons as a measure of secondary degeneration, and as the response of secondary degeneration to treatment. Therefore, secondary degeneration was quantified by injecting the dye immediately or 2 weeks after the primary injury, and calculating the additional loss of RGCs between the first and the second injections of the dye. The percentage of RGCs that had survived secondary degeneration was then calculated. The percentage of labeled RGCs (reflecting still-viable axons) was significantly greater in the retinas of the rats injected with anti-MBP T cells than in the retinas of the PBS-injected control rats (FIG. 2). In contrast, the percentage of labeled RGCs in the retinas of the rats injected with anti-OVA or anti-p277 T cells was not significantly greater than that in the control retinas. Thus, although the three T-cell lines accumulated at the site of injury, only the MBP-specific autoimmune T cells had a substantial effect in limiting the extent of secondary degeneration. Labeled RGCs of injured optic nerves of rats injected with PBS (FIG. 3A), with anti-p277 T cells (FIG. 3B) or with anti-MBP T cells were compared morphologically using micrographs (FIG. 3C).

### b 7.2.2 Clinical Severity of EAE

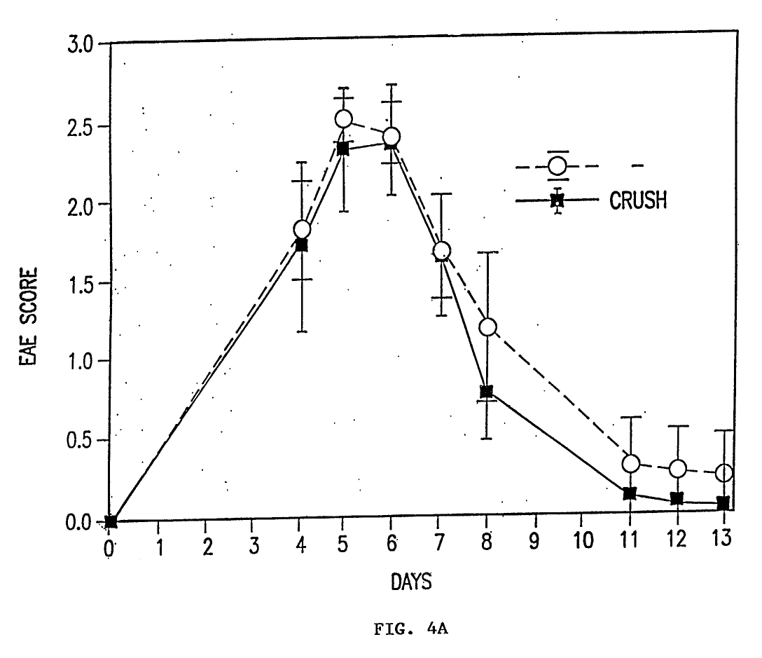

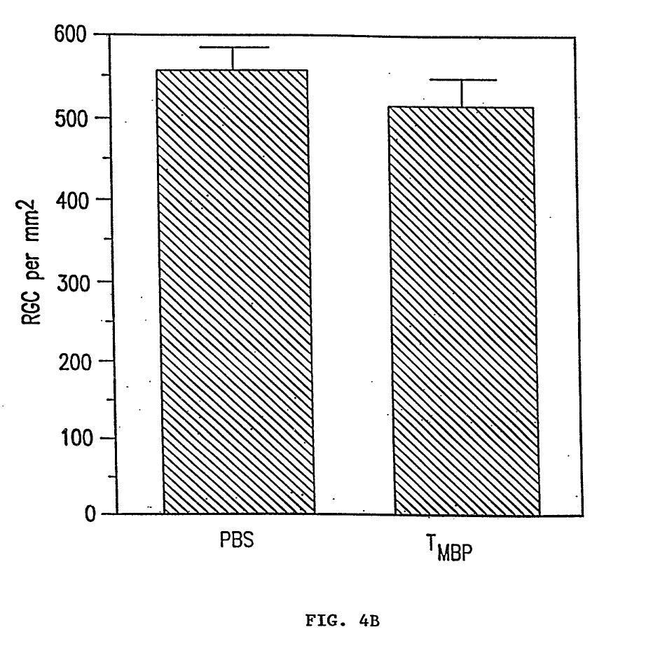

Animals were injected i.p. with 10⁷ T_{MBP} cells with or without concurrent optic nerve crush injury. The clinical course of the rats injected with the T_{MBP} cells was evaluated according to the neurological paralysis scale. Each group contained 5-9 rats. The functional autoimmunity of the injected anti-MBP T-cells was demonstrated by the development of transient EAE in the recipients of these cells. As can be seen in FIG. 4A, the course and severity of the EAE was not affected by the presence of the optic nerve crush injury.

### 7.2.3 Survival of RGCS in Non-Injured Nerves

Animals were injected i.p. with 10⁷ T_{MBP} cells or PBS. Two weeks later, 4-Di-10-Asp was applied to the optic nerves. After five days the retinas were excised and flat mounted. Labeled RGCs from five fields (located at approximately the same distance from the optic disk), in each retina were counted and their average number per are (mm²) was calculated.

As can be seen in FIG. 4B, there is no difference in the number of surviving RGCs per area (mm²) in non-injured optic nerves of rats injected with anti-MBP T-cells compared to in rats injected with PBS.

### 7.2.4 Neuroprotection by T-Cells Reactive to a Cryptic Epitope

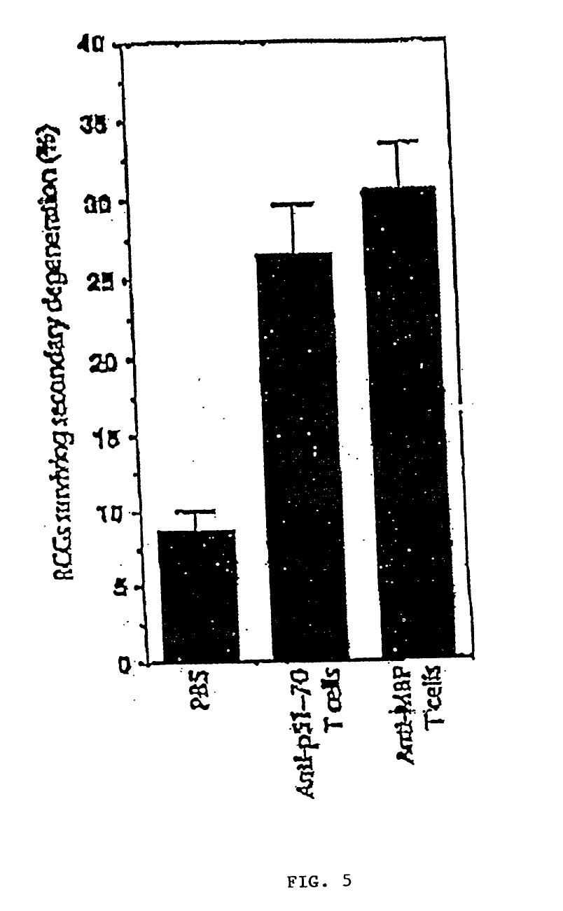

To determine whether the neuroprotective effect of the anti-MBP T cells is correlated with their virlence, the effect of T cells reactive to a ‘cryptic’ epitope of MBP, the peptide 51-70 (p51-70) was examined. ‘Cryptic’ epitopes activate specific T cells after an animal is immunized with the particular peptide, but not with the whole antigen (Mor, P. et al., 1995, J. Immunol. 155:3693-3699). The T-cell line reactive to the whole MBP and the T-cell line reactive to the cryptic epitope p51-70 were compared for the severity of the EAE they induced, and for their effects on secondary degeneration. In rats injected with the T-cell line reactive to the cryptic epitope, disease severity (as manifested by the maximal EAE score) was significantly lower than that in rats injected with the T-cell line reactive to the whole protein (Table 1). Whereas anti-MBP T cells caused clinical paralysis of the limbs, rats injected with the anti-p51-70 T cells developed only tail atony, not hind limb paralysis, and almost none showed weakness of the hind limbs. Despite this difference in EAE severity, the neuroprotective effect of the less virulent (anti-p51-70) T cells was similar to that of the more virulent (anti-MBP) T cells (FIG. 5). The percentage of RGCs surviving secondary degeneration in the retinas of rats injected with either of the lines was significantly higher than in the retinas of the PBS-injected rats. Thus, there was no correlation between the neuroprotective effect of the autoimmune T cells and their virulence. It is possible that the anti-p51-70 T cells encounter little antigen in the intact CNS, and therefore cause only mild EAE. Their target antigen may however become more available after injury, enabling these T cells to exert a neuroprotective effect.

TABLE 1
Anti-MBP and anti-p51-70 T cells
vary in pathogenicity

| T cell line | Clinical EAE | Mean max. score |
|---|---|---|
| Whole MBP | Moderate to severe | 2.00 ± 0.25 |
| p51-70 of MBP | Mild | 0.70 ± 0.2 |
| Immediately after optic nerve crush injury, Lewis rats were injected with activated anti-MBP T cells or anti-p51-70 T cells. The clinical course of EAE was evaluated according to the neurological paralysis scale. The mean maximal (max.) score ± s.e.m. was calculated as the average maximal score of all the diseased rats in each group. The table is a summary of nine experiments. Each group contains five to ten rats. Statistical analysis showed a | | |
| #significant difference between the mean maximal score of rats injected with anti-MBP T cells and that of rats injected with anti-p51-70 T cells (P = 0.039, Student's t-test). | | |

### 7.2.5 Electrophysiological Activity

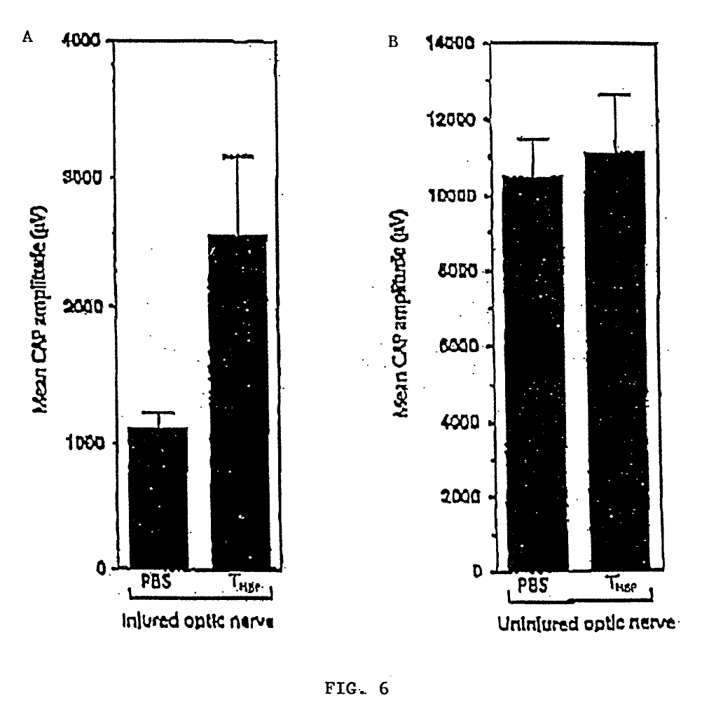

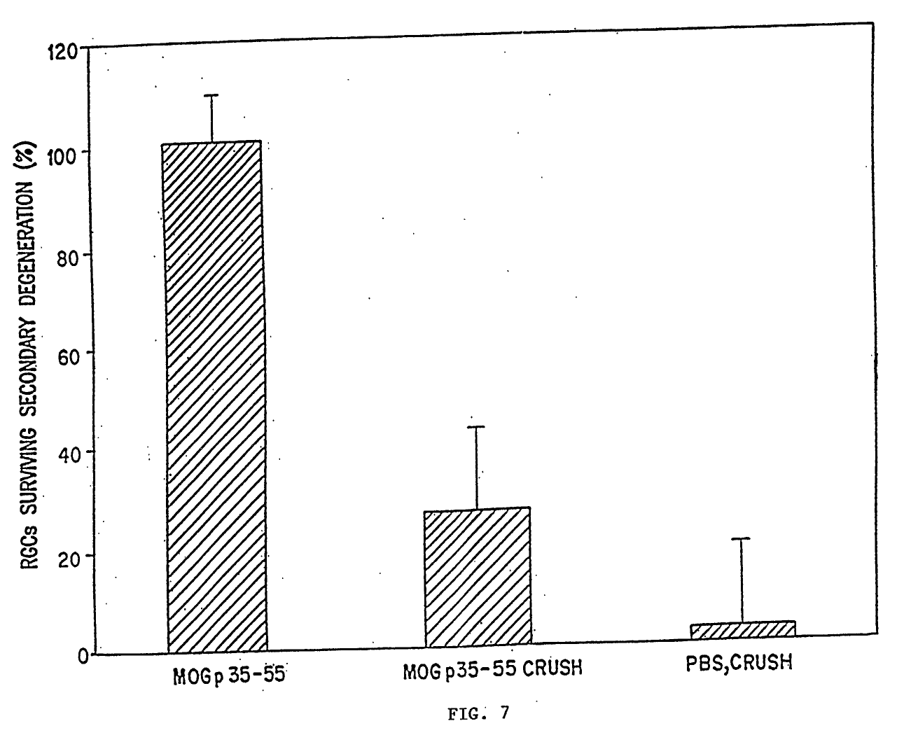

To confirm the neuroprotective effect of the anti-MBP T cells, electrophysiological studies were done. Immediately after optic nerve injury, the rats were injected intraperitoneally with PBS or with 1×10⁷ activated anti-MBP or anti-OVA T cells. The optic nerves were excised 7, 11 or 14 days later and the compound action potentials (CAPs), a measure of nerve conduction, were recorded from the injured nerves. On day 14, the mean CAP amplitudes of the distal segments recorded from the injured nerves obtained from the PBS-injected control rats were 33% to 50% of those recorded from the rats injected with the anti-MBP T cells. (FIG. 6A, Table 2). As the distal segment of the injured nerve contains both axons that escaped the primary insult and injured axons that have not yet degenerated, the observed neuroprotective effect could reflect the rescue of spared neurons, or a delay of Wallerian degeneration of the injured neurons (which normally occurs in the distal stump), or both. No effect of the injected anti-MBP T cells on the mean CAP amplitudes of uninjured nerves was observed (FIG. 6B, Table 2). It is unlikely that the neuroprotective effect observed on day 14 could have been due to the regrowth of nerve fibers, as the time period was too short for this.

The strong neuroprotective effect of the anti-MBP T cells seen on day 14 was associated with a significantly decreased CAP amplitude recorded on day 7 (Table 2). The anti-MBP T cells manifested no substantial effect on the uninjured nerve on day 7, indicating that the reduction in electrophysiological activity observed in the injured nerve on day 7 might reflect the larger number of T cells present at the injury site relative to the uninjured nerve (FIG. 1). The observed reduction in CAP amplitude in the injured nerve on day 7 reflected a transient reduction in conduction, which may have imposed a transient resting state in the injured nerve. This transient effect had not only disappeared, but was even reversed by day 14 (Table 2). Early signs of the neuroprotective effect could already be detected on day 11 in the rats injected with anti-MBP T cells (data not shown). In rats injected with anti-OVA T cells, no reduction in CAP amplitude on day 7 could be detected in either the injured or the uninjured nerves, and no neuroprotective effect was observed on day 14 (Table 2). Thus, it seems that the early reduction in CAP and the late neuroprotection shown specifically by the anti-MBP T cells are related.

TABLE 2
Transient reduction in electrophysiological
activity of the injured optic nerve induced by anti-MBP
T cells, followed by a neuroprotective effect

| | Uninjured optic nerve | | Injured optic nerve | |
|---|---|---|---|---|
| | Day 7 | Day 14 | Day 7 | Day 14 |
| Ratio (%) | 89.9 ± 9.4 | 101.2 ± 22.7 | 63.8* ± 14.9 | 243.1** ± 70.8 |
| T_{MPB}/PBS | (n = 22) | (n = 10) | (n = 17) | (n = 8) |
| Ratio (%) | 109.7 ± 13.2 | 92.5 ± 12.6 | 125.5 ± 24.4 | 107.3 ± 38.9 |
| T_{OVA}/PBS | (n = 1l) | (n = 3) | (n = 11) | (n = 4) |
| Immediately after optic nerve injury, rats were injected with PBS or with activated anti-MBP or anti-OVA T cells. After 7 or 14 days, the CAPs of injured and uninjured nerves were recorded. Ratios were calculated for uninjured nerves as (mean CAP of uninjured nerves from T cell-injected rats/mean CAP of uninjured nerves from PBS-injected rats) × 100, or for injured nerves as (mean CAP of injured nerves from T cell-injected rats/mean CAP of injured nerves from | | | | |
| #PBS-injected rats) × 100. The P value was calculated by comparing the logarithms of the normalized CAP amplitudes of nerves from PBS-injected rats and rats injected with T cells, using the unpaired Student's t-test, *P < 0.05; **P < 0.01 n = sample size. | | | | |

### 8. EXAMPLE

### Neuroprotective Effects of NS-Specific Antigen 8.1 Materials and Methods

Animals, crush injury of rat optic nerve, and retrograde labeling are described above in Sections 6 and 7. A peptide based on amino acids 35-55 of myelin/oligodendrocyte glycoprotein (MOG p35-55) was chemically synthesized at the Weizmann Institute, Israel.

### 8.1.1 Inhibition of Secondary Degeneration

Rats were injected intradermally in the footpads with MOG p35-55 (50 μg/animal) and IFA, or PBS ten days prior to optic nerve crush injury. Retinal ganglion cells were assessed two weeks after injury using retrograde labeling as described above. The number of RGCs in rats injected with PBS or MOG p35-55 was expressed as a percentage of the total number of neurons in rats injected with MOG p35-55 in the absence of crush injury.

### 8.2 Results

As shown in FIG. 7, the number of labeled retinal ganglion cells (indicating viable axons) was about 12.5 fold greater in animals injected with MOG p35-55 compared to in animals receiving PBS. 9. EXAMPLE

### Neuroprotective Effects of MBP Administered Orally

### 9.1 Materials and Methods

Animals, crush injury of rat optic nerve, and retrograde labeling of RGCs are described above in Sections 6 and 7.

### 9.1.1 Inhibition of Secondary Degeneration

Bovine MBP (Sigma, Israel) (1 mg/dose) was administered to rats by gavage using a blunt needle. MBP was administered 5 times, every third day, beginning 2 weeks prior to optic nerve crush injury. The number of RGCs in treated animals was expressed as a percentage of the total number of neurons in animals subjected to optic nerve crush injury but which did not receive MBP.

### 9.2 Results

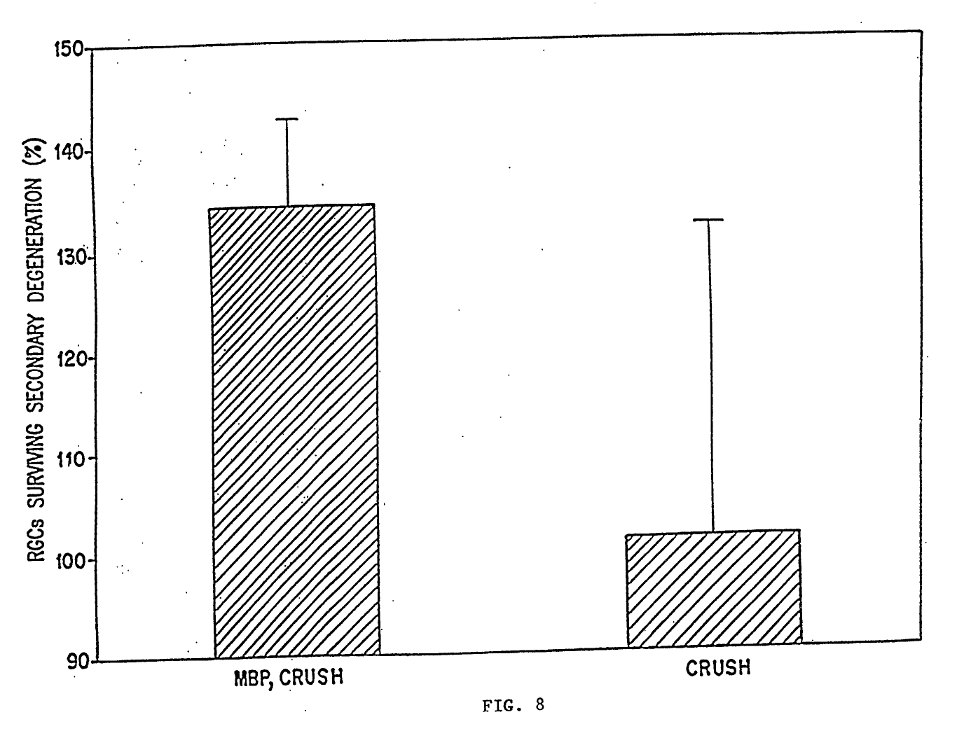

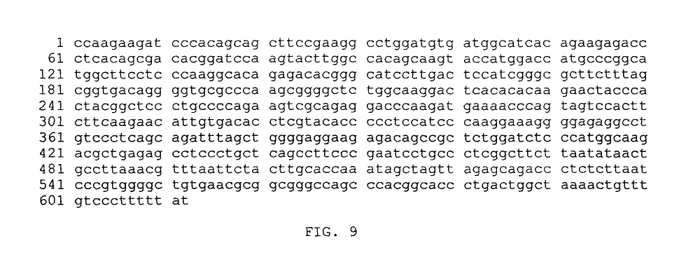

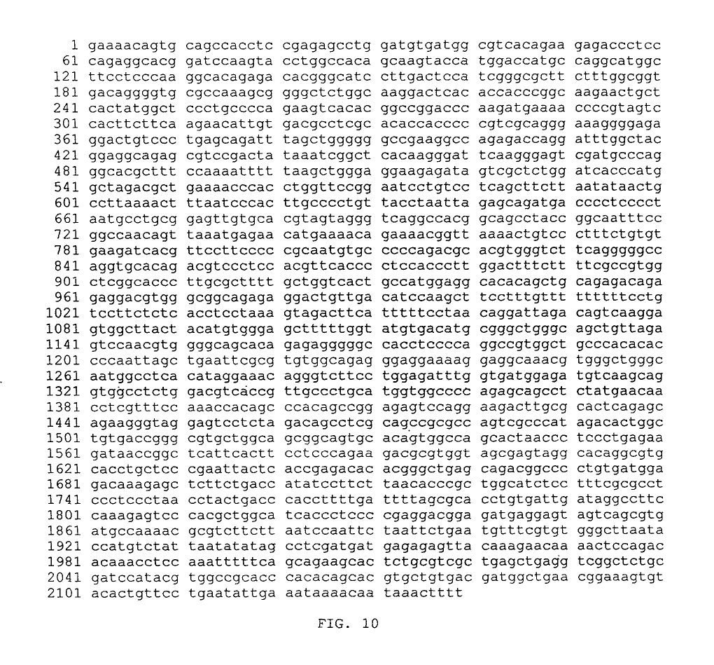

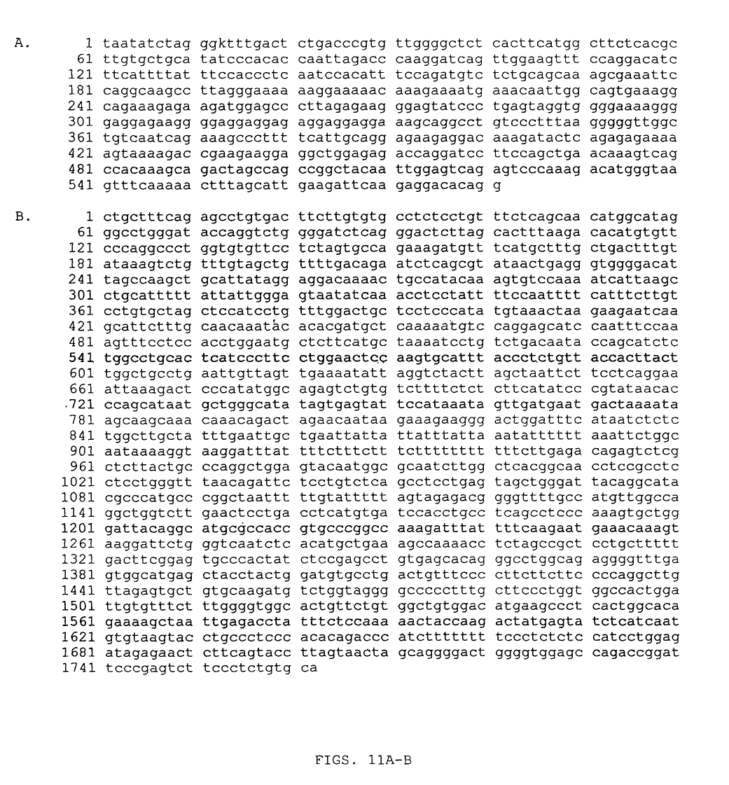

As shown in FIG. 8, the number of labeled RGCs was about 1.3 fold greater in animals treated with MBP compared to untreated animals.

### 10. DISCUSSION OF EXPERIMENTAL RESULTS

The results of the experiments described in Sections 6 and 7 show that activated T-cells accumulate at a site of injury in the CNS. Furthermore, the results also demonstrate that the accumulation of T-cells at the site of injury is a non-specific process, i.e., T-cells which accumulated at the site of injury included both T-cells which are activated by exposure to an antigen present at the site of injury as well as T-cells which are activated by an antigen not normally present in the individual.

The results of experiments described in Section 7 demonstrate that the beneficial effects of T-cells in ameliorating damage due to injury in the CNS are associated with a NS-specific self-antigen as illustrated by MBP. More specifically, the administration of non-recombinant T-cells which were activated by exposure to an antigen which can cause autoimmune disease (T_{MBP}), rather than aggravating the injury, led to a significant degree of protection from secondary degeneration. Thus, activating T-cells by exposure to a fragment of a NS-specific antigen was beneficial in limiting the spread of injury in the CNS. The present findings show that secondary degeneration can be inhibited by the transfer into the individual of non-recombinant T-cells which recognize a NS-specific self antigen which is present at a site of injury. The T-cells may recognize cryptic or non-pathogenic epitopes of NS-self antigens.

In addition, the studies described in Sections 8 and 9 show that activation of T-cells by administering an immunogenic antigen (e.g. MBP) or immunogenic epitope of an antigen (e.g. MOG p35-55), may be used for preventing or inhibiting secondary CNS degeneration following injury.

The present invention is not to be limited in scope by the exemplified embodiments, which are intended as illustrations of single aspects of the invention. Indeed, various modifications of the invention in addition to those shown and described herein will become apparent to those skilled in the art from the foregoing description and accompanying drawings. Such modifications are intended to fall within the scope of the appended claims.

All publications cited herein are incorporated by reference in their entirety.

What is claimed is:

1. A method for preventing or inhibiting axonal degeneration in the central nervous system or peripheral nervous system comprising administering to a human in need thereof:
(a) non-recombinant, NS-specific antiself activated T-cells;
(b) a NS-specific antigen;
(c) a peptide derived from a NS-specific antigen;
(d) a nucleotide sequence encoding a NS-specific antigen;
(e) a nucleotide sequence encoding a peptide derived from a NS-specific antigen; or (f) any combination of (a)-(e), to ameliorate the effects of injury or disease.

2. A method for promoting nerve regeneration in the central nervous system or peripheral nervous system comprising administering to a human in need thereof:
(a) non-recombinant, NS-specific antiself activated T-cells;
(b) a NS-specific antigen;
(c) a peptide derived from a NS-specific antigen;
(d) a nucleotide sequence encoding a NS-specific antigen;
(e) a nucleotide sequence encoding a peptide derived from a NS-specific antigen; or
(f) any combination of (a)-(e), to ameliorate the effects of injury or disease.

3. The method according to claim 1 or 2 in which said injury comprises blunt trauma, penetrating trauma, hemorrhagic stroke, ischemic stroke, or damages caused by surgery.

4. The method of claim 1 or 2 in which said disease is Diabetic neuropathy, senile dementia, Alzheimer's disease, Parkinson's Disease, facial nerve (Bell's) palsy, glaucoma, Huntington's chorea, amyotrophic lateral sclerosis, non-arteritic optic neuropathy, or vitamin deficiency.

5. The method of claim 1 or 2 in which said disease is not an autoimmune disease or a neoplasm.

6. The method of claim 1 or 2 in which said peptide derived from a NS-specific antigen is an immunogenic epitope or a cryptic epitope.

7. The method according to claims 1 or 2 in which said NS-specific antigen is administered intravenously, intraperitoneally, intramuscularly, subcutaneously, orally, intranasally, vaginally, rectally, intraocularly, intrathecally, intradermally, or buccally.

8. The method according to claim 1(a), 1(c), 1(d), 1(e), 2(a), 2(c), 2(d), or 2(e), further comprising administering to a human in need thereof a NS-specific antigen.

9. The method according to claim 8 in which said NS-specific antigen is administered before or after administration of the composition according to claim 1(a), 1(c), 1(d), 1(e), 2(a), 2(c) or 2(e).

10. The method according to claim 8 in which said NS-specific antigen is administered concurrently with administration of the composition according to claim 1(a), 1(c), 1(d), 1(e), 2(a), 2(c) or 2(e).

11. The method according to claim 1 or 2 in which said T-cells are attenuated.

12. The method according to claim 1 or 2 in which said T-cells are autologous or allogeneic.

13. The method according to claim 1 or 2 in which the NS-specific antigen or peptide derived therefrom is myelin basic protein, myelin oligodendrocyte glycoprotein, proteolipid protein, myelin-associated glycoprotein, S-100, β-amyloid, Thy-1, P0, or P2.

14. The method according to claim 1d or 2d in which the nucleotide sequence is depicted in FIG. 9, FIG. 10, FIG. 11(A-F), FIG. 12, FIG. 13, or FIG. 14.

15. The method according to claim 1 or 2 in which the NS-specific antigen comprises the amino acid sequence of FIG. 15, FIG. 16, or FIG. 17.
